(19) United States
(12) Patent Application Publication
(10) Pub. No.: US 2025/0144369 A1
(43) Pub. Date: May 8, 2025

(54) MAGNETIC BREAKAWAY FOR MEDICAL DEVICE

(71) Applicant: CANON U.S.A., INC., Melville, NY (US)

(72) Inventor: Zachary Hamilton Haubert, Somerville, MA (US)

(21) Appl. No.: 18/837,407

(22) PCT Filed: Feb. 9, 2023
(86) PCT No.: PCT/US2023/062326

§ 371 Date: Aug. 9, 2024

Related U.S. Application Data
(60) Provisional application No. 63/308,713, filed on Feb. 10, 2022.
(60) Provisional application No. 63/314,940, filed on Feb. 28, 2022.

(51) Int. Cl. A61M 25/01; A61M 25/00; A61M 39/10

(52) U.S. Cl. CPC A61M 25/0147; A61M 25/0074; A61M 39/10; A61M 2039/1061; A61M 2205/0216; A61M 2205/0233; A61M 2205/025; A61M 2205/0266; A61M 2205/10; A61M 2205/332; A61M 2205/3327; A61M 2205/50

(57) ABSTRACT

A breakaway apparatus (100) according to some embodiments can include an actuator, a medical device, and a breakaway connection coupling the actuator and the medical device, the breakaway connection having an actuator portion on the actuator and a medical device portion on the medical device. The actuator portion can include an actuator support (110), an actuator side ferromagnetic block (116), and a slider element (126). The medical device portion can include a medical device support, a medical device side ferromagnetic block, and a slider element. At least one of the actuator side ferromagnetic block and the medical device side ferromagnetic block includes a magnet, and the actuator is coupled to the medical device by a magnetic coupling force. The actuator will decouple from the medical device when a decoupling force is greater than a predetermined amount.

### CROSS-REFERENCE TO RELATED APPLICATIONS

This application claims the benefit of priority from U.S. Provisional Application No. 63/308,713, filed Feb. 10, 2022, and U.S. Provisional Application No. 63/314,940, filed Feb. 28, 2022, each of which is hereby incorporated by reference herein in their entirety.

### TECHNICAL FIELD

The present disclosure generally relates to medical connectors and, more particularly, to a magnetic breakaway for a medical device.

### BACKGROUND ART

Minimally-invasive imaging, diagnostic, or therapeutic devices find use in image guided therapy to look inside a body. A flexible medical device or tool, such as a catheter, endoscope, colonoscope, bronchoscope, ablation device, or the like can carry out these types of medical procedures, where the medical device is inserted into a patient's body and an instrument is passed through the tool to examine or treat an area inside the body. A bronchoscope is an endoscopic instrument to view inside the airways of a patient. Catheters and other medical tools can be inserted through a tool channel in the bronchoscope to provide a pathway to a target area in the patient for diagnosis, treatment, or the like.

A continuum robot, robotic and/or snake catheter assembly are exemplary medical arrangements or configurations that can implement the flexible medical device to carry out the medical procedures. These medical configurations typically have a rotational drive assembly to impart rotational movement to a guide wire of a steerable catheter, endoscope, or other medical device. The drive assembly is releasably connected to the catheter and a breakaway mechanism can be used so the drive assembly disconnects from the catheter in response to a breakaway force.

A basic snake catheter assembly, for example, generally includes a steerable catheter actuated with push-pull wires, a motorized actuator for driving catheter tip motions through the push-pull wires, and a controller that translates user/software commands into actuator motion.

Steerable catheters with push-pull wires have advantages over conventional steerable catheters to generate a large bending moment without contraction of the catheter along the axial direction. However, by actuating wires with both push and pull directions, tensile and contraction forces on the wires can lead to modes of failure including wire anchor fracturing (wire anchors being the bonding mechanism between the wire and the catheter tip), wire prolapse and protrusion, and excessive lateral bending force to internal tissues such as lung tissues or the like.

Breakaway mechanisms generally have two stages including a compressive breakaway mechanism and a tensile breakaway mechanism, as shown in FIG. 17. In the compressive breakaway mechanism, a push acts on the center of a magnet. There are issues regarding rigidity, force density, and bandwidth that present problems for these types of breakaway mechanisms.

As to rigidity, for the compressive device to work properly, the mechanism relies on a plunger that encapsulates the magnet. The purpose of this part is to create a connection between the magnet and the guide wire. Certain types of breakaway mechanisms rely on a tightly controlled fit between two or more parts. Thus, the parts should be manufactured to exacting processes and material standards. These parts are non-magnetic and extremely thin so they do not attenuate the breakaway force. High failure rates can be caused by problems such as material defects, tooling errors, and poor process control. However, this makes the parts difficult to manufacture as common manufacturing techniques generally provide a minimum 0.5 mm wall thickness. Breakaway mechanisms normally require the thickness to be close to 0.1 mm with a ¼ inch outer diameter magnet. This means an overall diameter and volume of the breakaway mechanism would have to increase to obtain adequate breakaway force. Also, due to the thin-walled requirement of the plunger, it also means that the part would not be very rigid and will break away under cyclic loading.

With respect to force density, aside from not being able to generate enough force due to the wall thickness manufacturing restriction, there is also an issue of lost ferrous metal volume since the plunger goes through the center of the mechanism.

For bandwidth, since there are two stages and two connections it increases the complexity and spring-damper effect of the system. This can have effects of the bandwidth of the system.

It would be beneficial to mitigate these concerns, improve safety and address issues regarding rigidity, force density, and bandwidth.

### SUMMARY

The present disclosure advantageously provides magnetic breakaway configurations that improve safety and address issues regarding rigidity, force density, and bandwidth.

A breakaway apparatus according to some embodiments includes an actuator, a medical device, and a breakaway connection coupling the actuator and the medical device, the breakaway connection having an actuator portion on the actuator and a medical device portion on the medical device. The actuator portion can include an actuator support, an actuator side ferromagnetic block, and a slider element. The medical device portion can include a medical device support, a medical device side ferromagnetic block, and a slider element. At least one of the actuator side ferromagnetic block and the medical device side ferromagnetic block includes a magnet, and the actuator is coupled to the medical device by a magnetic coupling force. The actuator will decouple from the medical device when a decoupling force is greater than a predetermined amount.

The actuator portion can include a spacer attached to the actuator side ferromagnetic block and facing the medical device ferromagnetic block, the spacer defining a gap between the actuator side ferromagnetic block and the medical device ferromagnetic block. Both the actuator side ferromagnetic block and the medical device side ferromagnetic block can include a magnet.

The slider element in the actuator portion and the slider element in the medical device portion can be a single slider element that is connected to both the actuator support and the medical device support.

The actuator can include a motor, a drive, or a motorized drive assembly. The actuator can include a rotational drive assembly to impart rotational movement to the plurality of drive wires. The plurality of drive wires can include a monolithic material wire configured for both push and pull motions.

The medical device can be a catheter, endoscope, colonoscope, bronchoscope, or ablation device. The medical device can be a steerable catheter with distal and proximal ends and a plurality of driving wires. The steerable catheter can include a plurality of bendable sections located at a distal end of the steerable catheter, one of the plurality of drive wires passing through each bendable section. The driving wires are push-pull wires can have a distal end attached to a distal end of one of the plurality of bendable sections and a proximal end attached to the medical device portion.

A tensile force breakaway mechanism can engage at a tensile threshold force presented on at least one of the plurality of driving wires, which is equal to or greater than a predetermined value to stop tensile force transmission through the actuator portion to the steerable catheter. The tensile force breakaway mechanism can be in the steerable catheter. The plurality of driving wires can include super-elastic material in the form of nickel titanium alloy. The plurality of driving wires reach a super-elastic plateau of the super-elastic material in a stress-strain relationship at the tensile threshold force, and an elongation of the plurality of driving wires in the super-elastic plateau state is configured to stop the tensile force transmission. The plurality of driving wires in the super-elastic plateau state can have a maximum elongation equal to or greater than a predetermined operation stroke distance of the plurality of driving wires that corresponds to a maximum operation stroke distance and the maximum elongation.

A compression force breakaway mechanism can engage at a compression threshold force on at least one of the plurality of driving wires to stop compression force transmission via the at least one driving wire from the actuator to the steerable catheter. The compression force breakaway mechanism can be in the actuator. The compression force breakaway mechanism can include permanent magnets.

The slider element on the actuator portion and the slider element on the medical device portion together constrain the plurality of drive wires in at least one dimension. The slider element on the actuator portion and the slider element on the medical device portion together constrain the plurality of drive wires in five dimensions.

The breakaway apparatus can include at least one force sensor coupled to the steerable catheter or medical device portion to measure push and pull forces on the plurality of driving wires, at least one motor to push or pull the plurality of driving wires based on operation commands, and a controller to issue the operation command to the at least one motor. The controller can issue the commands based on metrics computed with force measurements from the force sensors to stop increasing the push or pull force on the push-pull wires. The actuator can exert linear force provided by a motor. The linear force can act through the breakaway apparatus exerting itself onto the medical device.

The actuator portion and the medical device portion can be offset from each other in a direction of the magnetic coupling force. An actuator pusher can be coupled to the actuator. The medical device can include a medical device wire pusher. The actuator ferromagnetic block and the medical device ferromagnetic block are configured to permeate magnetic flux and create a bond between each other.

A system according to some embodiments can include an actuator, a steerable device having a plurality of driving wires, and a plurality of breakaway connections coupling the actuator and the steerable device, each breakaway connection having an actuator portion on the actuator and a steerable device portion on the medical device.

The actuator portions can each include an actuator support, an actuator side ferromagnetic block, and a slider element. The steerable device portions can each include device support, a steerable device side ferromagnetic block, and a slider element. The at least one of the actuator side ferromagnetic block and the device side ferromagnetic block can include a magnet. A tensile force breakaway mechanism can engage at a tensile threshold force on at least one of the plurality of driving wires which is equal to or greater than a predetermined value to stop tensile force transmission through the actuator portion to the steerable device. A compression force breakaway mechanism can engage at a compression threshold force on at least one of the plurality of driving wires to stop compression force transmission through the actuator to the steerable device. The system can include at least six breakaway connections and the steerable device can include at least three driving wires. The actuator is coupled to the medical device by a magnetic coupling force, and the actuator will decouple from the steerable device when a decoupling force is greater than a predetermined amount.

Both the actuator side ferromagnetic block and the steerable device side ferromagnetic block can include a magnet. The slider element in the actuator portion and the slider element in the steerable device portion can be a single slider element that is connected to both the actuator support and the steerable device support. The actuator can include a motor, a drive, or a motorized drive assembly.

The system can also include any combination of additional features as described above for the breakaway apparatus.

Further features of the present disclosure will become apparent from the following description of exemplary embodiments with reference to the attached drawings, where like structure is indicated with like reference numerals.

### BRIEF DESCRIPTION OF THE DRAWINGS

FIG. 1 illustrates a breakaway connection with a magnet according to an exemplary embodiment.

FIG. 2 illustrates a slider mechanism supporting some features of the breakaway connection of FIG. 1 in an engaged orientation.

FIG. 3 illustrates the slider mechanism of FIG. 2 in a disengaged orientation.

FIG. 4 illustrates a breakaway connection with a magnet and a spacer according to an exemplary embodiment.

FIG. 5 illustrates a slider mechanism supporting some features of the breakaway connection of FIG. 4 in an engaged orientation.

FIG. 6 illustrates the slider mechanism of FIG. 5 in a disengaged orientation.

FIG. 7 illustrates a breakaway connection with two magnets and a spacer according to an exemplary embodiment.

FIG. 8 illustrates components of the breakaway connection of FIG. 7 arranged on a slider mechanism in an engaged orientation.

FIG. 9 illustrates the breakaway connection of FIG. 8 in a disengaged orientation.

FIG. 10 illustrates a diagram showing a pull test data of a single magnet versus a double magnet.

FIG. 11 illustrates a tensile test diagram according to some embodiments.

FIG. 12 illustrates a force management diagram according to some embodiments.

FIG. 13 illustrates another force management diagram according to some embodiments.

FIG. 14 illustrates a stress strain curve for super-elastic Nitinol.

FIG. 15 illustrates a force displacement curve from tested Nitinol wire.

FIG. 16 illustrates a breakaway connection according to an exemplary embodiment.

FIG. 17 illustrates a breakaway connection according to an exemplary embodiment.

FIG. 18 illustrates a breakaway connection according to an exemplary embodiment.

FIG. 19 illustrates a slide mechanism for implementing a breakaway connection according to some embodiments.

FIG. 20 illustrates a medical apparatus according to some embodiments.

FIG. 21 illustrates a block diagram of the medical apparatus of FIG. 20.

FIG. 22 illustrates a block diagram of a controller according to some embodiments.

FIGS. 23A and 23B illustrate a catheter according to some embodiments.

FIG. 24 illustrates a breakaway apparatus according to the prior art.

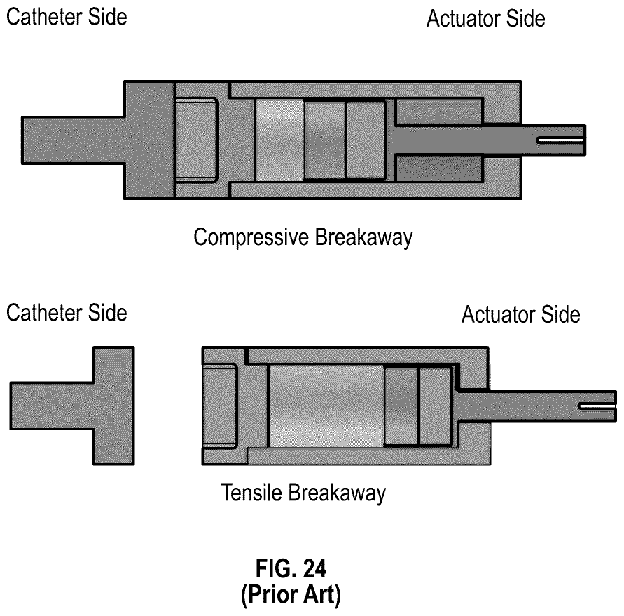

### DESCRIPTION OF THE EMBODIMENTS

Various exemplary embodiments, features, and aspects of the disclosure that relate to magnetic breakaway configurations for a medical device that improve safety and address issues regarding rigidity, force density, and bandwidth will be described below with reference to the drawings that may have different characteristics, advantages, disadvantages, performance parameters, or the like. The present disclosure is not limited to any particular configuration.

In the following embodiments, magnetic breakaway configurations are described that functionally interact with a medical device. The medical device is preferably a flexible device and can be a catheter, endoscope, colonoscope, bronchoscope, ablation device, or the like. The medical device can be used with various medical arrangements or configurations, such as a continuum robot, robotic or snake catheter assembly, or the like, to carry out medical procedures where the medical device is inserted into a patient's body and an instrument is passed through the tool to examine or treat an area inside the body. These medical arrangements have an actuator with a motor or drive configured as a rotational drive assembly to impart rotational movement to a driving wire or guidewire of a steerable catheter, endoscope, or other medical device. The drive wire can include one or more push-pull wires, drive wires, support wires, or other types of wires, and the drive assembly can drive bendable sections of the catheter by pushing and/or pulling the driving wires in a push/pull or insertion/pulling-out direction. Breakaway configurations according to some embodiments can be used so the drive assembly is releasably connected to the medical device and disconnects from the medical device in response to a coupling or breakaway force greater than a predetermined amount.

A steerable catheter apparatus, configuration or arrangement is an exemplary configuration of the medical device according to some embodiments and can include a steerable catheter with a catheter tip actuated with push-pull wires, a motorized actuator for driving the catheter tip motions through the push-pull wires, a controller that translates user/software commands into actuator motion, and can include other elements or components. The catheter can have a proximal end near the actuator and a distal end near the catheter tip. Variations or other configurations of the medical device are within the scope of the present disclosure and are not limited to these arrangements.

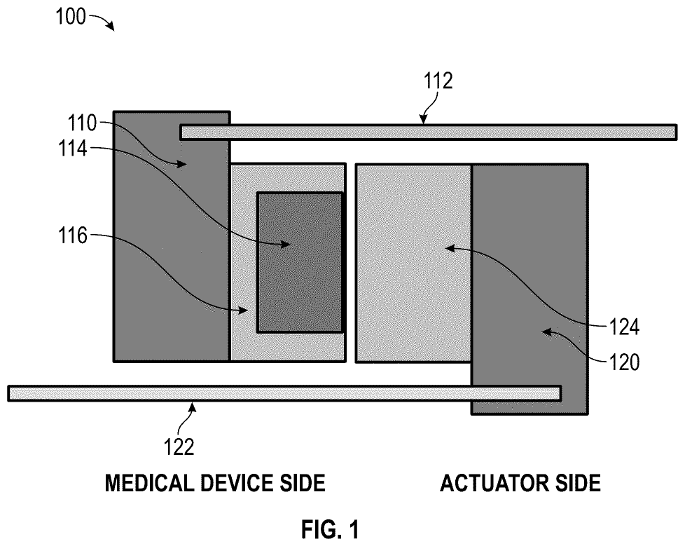

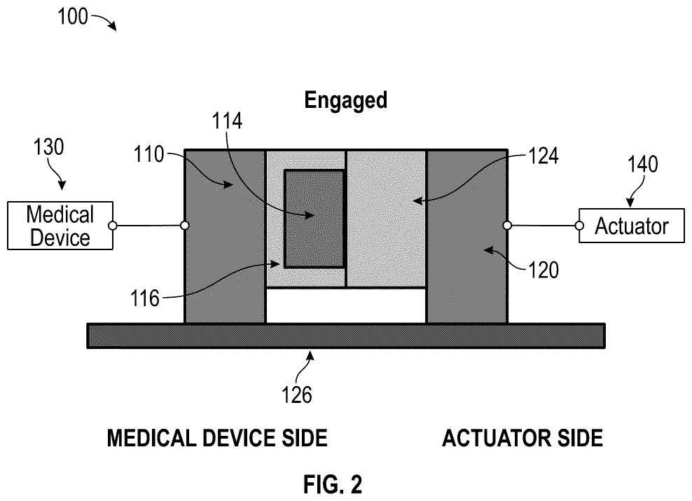

FIG. 1 shows a breakaway apparatus 100 with a magnet according to an exemplary embodiment. The breakaway apparatus 100 that includes a magnetic breakaway connection with a medical device side and an actuator side. The breakaway apparatus 100 interconnects a medical or treatment device 130 on the medical device side with an actuator 140 configured as a motor, drive, motorized drive assembly, or the like on the actuator side (see FIG. 2). With reference to the drawings and throughout the present application, reference is made to the “medical device side” and the “actuator side”, where the medical device components are on the left side of the pages, and the actuator components (motors) are on the right side of the pages. As used herein, reference is occasionally and interchangeably made to “medical device” or “treatment”, e.g. “treatment side” or “treatment device” rather than “medical device side” or “medical device”, and correspond to the same general features. The breakaway components hook around each other so the actuator side magnet block is on the left of the medical device side block.

The drive assembly is releasably connected to the medical device and disconnects from the medical device in response to a coupling or breakaway force that is greater than or equal to a desired or predetermined amount. The drive assembly is configured as a rotational drive assembly to impart rotational movement to at least one drive wire of the medical device. The drive wire can be a monolithic material wire configured for both push and pull motions, and can include one or more drive wires, push-pull wires, support wires, tendons, strands or other types of wires, and the drive assembly can drive bendable sections of the medical device by pushing and/or pulling the driving wires in a push/pull or insertion/pulling-out direction. The wire(s) are preferably made out of a single material and not a braid or coil. The medical device is preferably a flexible device and can be a catheter, endoscope, colonoscope, bronchoscope, ablation device, or other type of medical device.

The actuator side include one or more of an actuator support 110, an actuator pusher 112, a magnet 114, a ferromagnetic block 116, and can include other elements or components. The actuator support 110 and actuator pusher 112 can be configured together as the same entity according to some embodiments. The medical device side includes one or more of a medical device support 120, a medical device wire pusher 122, a ferromagnetic block 124, and can include other elements or components. The magnet 114 can be contained within the ferromagnetic block 116 and has a coupling force capability that depends on characteristics including the material, shape, configuration, weight, temperature, composition, workload mass, and can include a variety of other factors. The actuator side is coupled to the medical device side by a magnetic coupling force that is greater than or equal to a predetermined amount.

The actuator pusher 112 exerts linear force that is provided by a motor or motorized drive assembly. The force acts through the breakaway connection 100 and exerts itself onto the medical device wire pusher 122. The actuator support 110 and the medical device support 120 present on both sides of the breakaway connection 100 guide the force from each respective pusher 112, 122 to the breakaway components. The actuator support 110 and the medical device support 120 are preferably rigid to handle forces and can be made out of non-ferrous metal such as aluminum or the like. The medical device generally has a small pitch circle for drive wires so the magnetic force should not exceed a predetermined level large enough to fit axially among the wires without interfering with each other. The breakaway connection 100 is offset and that is why the supports 110, 120 exist for the actuator pusher side and for the medical device side. The ferromagnetic blocks 116, 124 are made of strong metal material having predetermined strength criteria, e.g. tensile strength, compressive strength, yield strength, impact strength, etc., such as steel, steel alloys, tungsten, titanium, chromium, or the like, e.g. 1018 steel, etc., and serve to permeate magnetic flux and create a bond between the two sides, the actuator support 110 and the medical device support 120, out of magnetic force that is characterized as being equal to a predetermined or desired coupling force.

The breakaway connection 100 can have a compressive breakaway mechanism located in the actuator side or proximal end of the medical device that decouples the actuator from the drive wire once a threshold force is passed or has occurred. This breakaway mechanism is a passive mechanical assembly that does not rely on electrical power to operate. Specifically, the breakaway mechanism 100 with the magnet 114 contained within the ferromagnetic block 116 can decouple the actuator from the drive wire once the force is greater or equal to the threshold force.

According to some embodiments, breakaway configurations can include a linear motion device with a plurality of guide rails laid in parallel and straight at a predetermined distance. The linear motion device can include a plurality of sliders arranged on each side of the plurality of guide rails and movable on the guide rails. A movable part can be bridged over all of the plurality of guide rails and can be supported by the plurality of sliders and be movable along the plurality of guide rails.

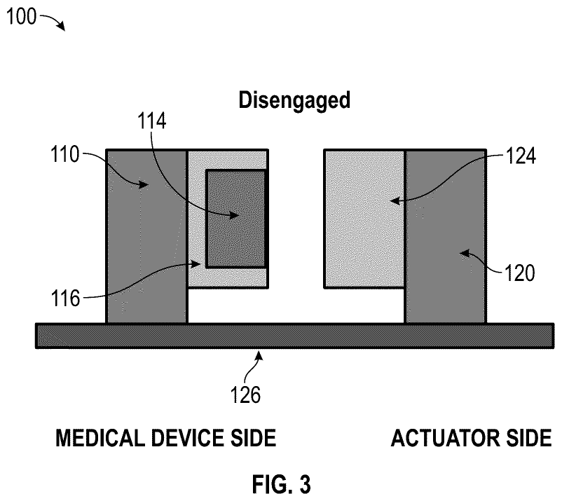

FIG. 2 illustrates a slider 126 that is a slider mechanism supporting some features of the breakaway connection 100 of FIG. 1 in an engaged orientation. FIG. 3 illustrates the breakaway connection 100 of FIG. 2 in a disengaged orientation.

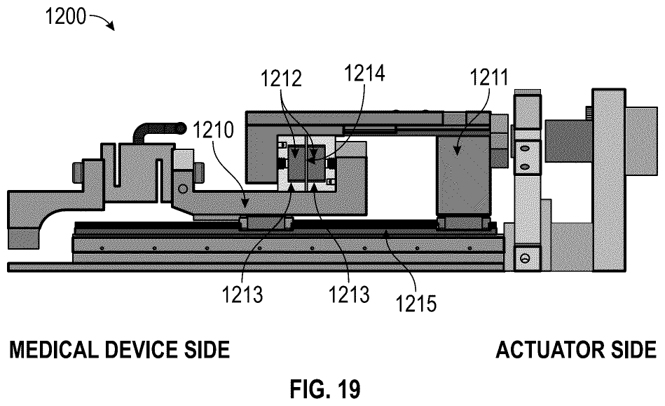

The slider 126 of FIGS. 2 and 3 is an exemplary linear motion device configured as a slide mechanism for implementing a breakaway connection according to an exemplary embodiment. The breakaway apparatus 100 interconnects the medical device 130 on the medical device side with the actuator 140 configured as a motor, drive, motorized drive assembly, or the like on the actuator side. The slider 126 is a linear guide rail and can have an aluminum pusher on the medical device side and an aluminum pusher on the actuator side. The magnet 114 can be, for example, an N52 NdFeB magnet or other type of magnet. Linear guide rails, guideways, and slides are mechanical systems composed of rails and bearings that support and move physical loads along a linear path with a low coefficient of friction. These are generally considered as rolling element or plane bushing types. Common types of guideways and bearings include profiled (square) rails with recirculating ball bearing blocks, guideways for roller bearings, and round rails with recirculating ball bushings or plane bushings. The slides can be saddle, cantilever, or other types of slides. The slider 126 can have a plurality of guide rails laid in parallel and straight at a predetermined distance. A plurality of sliders can be arranged on each of the plurality of guide rails and movable on the guide rails, and a movable part that is bridged over all of the guide rails and is supported by the plurality of sliders and movable along the plurality of guide rails. See FIG. 19 for more details about slide mechanisms according to some embodiments.

A breakaway apparatus according to some embodiments can include an actuator, a medical device, and the breakaway connection 100 coupling the actuator 140 and the medical device 130, the breakaway connection 100 having an actuator portion on the actuator 140 and a medical device portion on the medical device 130. The actuator portion can include the actuator support 110, the actuator side ferromagnetic block 116, and the slider element 126. The medical device portion can include the medical device support 120, the medical device side ferromagnetic block 124, and the slider element 126. At least one of the actuator side ferromagnetic block 116 and the medical device side ferromagnetic block 124 includes a magnet 114, and the actuator is coupled to the medical device by a magnetic coupling force. The actuator will decouple from the medical device when a decoupling force is greater than a predetermined amount.

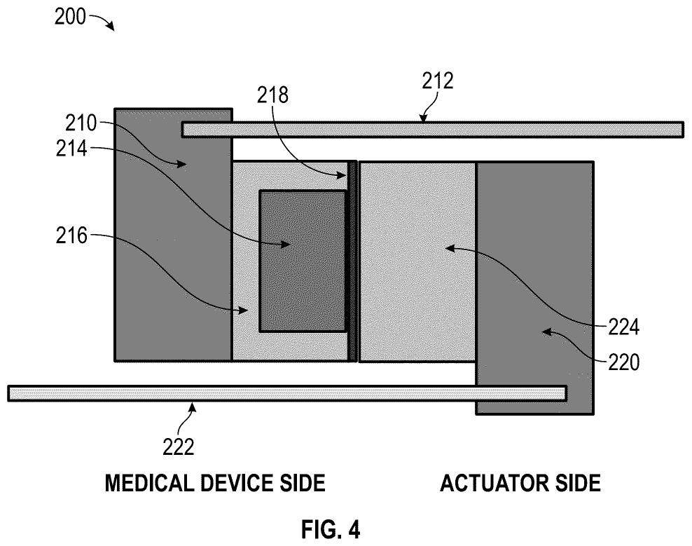

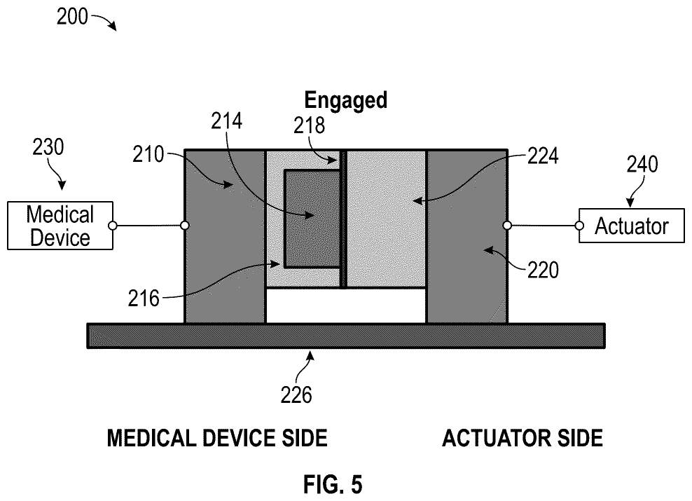

FIG. 4 shows a breakaway apparatus 200 with a magnet and a spacer according to an exemplary embodiment. The breakaway apparatus 200 interconnects a medical or treatment device 230 on the medical device side with an actuator 240 configured as a motor, drive, motorized drive assembly, or the like on the actuator side (see FIG. 5). The drive assembly is releasably connected to the medical device and disconnects from the medical device in response to a coupling force that is greater than or equal to a desired or predetermined amount. The drive assembly is configured as a rotational drive assembly to impart rotational movement to at least one drive wire of the medical device. The drive wire can be a monolithic material wire configured for both push and pull motions, and can include one or more drive wires, push-pull wires, support wires, tendons, strands or other types of wires, and the drive assembly can drive bendable sections of the medical device by pushing and/or pulling the driving wires in a push/pull or insertion/pulling-out direction. The wire(s) are preferably made out of a single material and not a braid or coil. The medical device is preferably a flexible device and can be a catheter, endoscope, colonoscope, bronchoscope, ablation device, or other type of medical device. The breakaway connection 200 is similar to FIG. 1 but also includes a spacer or shim 218.

The actuator side includes one or more of an actuator support 210, an actuator pusher 212, a magnet 214, a ferromagnetic block 216, the spacer 218, and can include other elements or components. The actuator support 210 and actuator pusher 212 can be configured together as the same entity according to some embodiments. The medical device side includes one or more of a medical device support 220, a medical device wire pusher 222, a ferromagnetic block 224, and can include other elements or components. The magnet 214 can be contained within the ferromagnetic block 216 and has a coupling force capability that depends on characteristics including the material, shape, configuration, weight, temperature, composition, workload mass, and can include a variety of other factors.

Since the coupling force is dependent on the gap between the two ferromagnetic blocks 216, 224, the spacer 218 is used to maintain a desired gap in order to achieve a predetermined or desired coupling force. The spacer 218 or shim can be made from plastic or a non-ferrous metal such as aluminum or the like. A thick spacer will result in a lower coupling force while a thinner spacer will result in a higher coupling force.

The actuator pusher 212 exerts linear force that is provided by a motor or motorized drive assembly. The force acts through the breakaway connection 200 and exerts itself onto the treatment wire pusher 222. The actuator support 210 and the treatment support 220 present on both sides guide the force from each respective pusher 212, 222 to the breakaway components. The actuator support 210 and the treatment support 220 are preferably rigid to handle forces and can be made out of non-ferrous metal such as aluminum or the like. The medical device generally has a small pitch circle for drive wires so the magnetic force should not exceed a predetermined level large enough to fit axially among the wires without interfering with each other. The breakaway is offset and that is why the supports 210, 220 exist for the actuator pusher side and for the treatment side. The ferromagnetic blocks 216, 224 are made of strong metal material having predetermined strength criteria, e.g. tensile strength, compressive strength, yield strength, impact strength, etc., such as steel, steel alloys, tungsten, titanium, chromium, or the like, e.g. 1018 steel, etc., and serve to permeate magnetic flux and create a bond between the two sides, the actuator support 210 and the treatment support 220, out of magnetic force that is characterized as being equal to a predetermined or desired coupling force.

The breakaway connection 200 can have a compressive breakaway mechanism located in the actuator side or proximal end of the medical device that decouples the actuator from the drive wire once a threshold force is passed or has occurred. This breakaway mechanism is a passive mechanical assembly that does not rely on electrical power to operate. Specifically, the breakaway mechanism 200 with the magnet 214 contained within the ferromagnetic block 216 can decouple the actuator from the drive wire once the threshold force is passed.

A breakaway apparatus according to some embodiments can include an actuator, a medical device, and the breakaway connection 200 coupling the actuator and the medical device, the breakaway connection 200 having an actuator portion on the actuator and a medical device portion on the medical device. The actuator portion can include the actuator support 210, the actuator side ferromagnetic block 216, and the slider element 226. The medical device portion can include the medical device support 220, the medical device side ferromagnetic block 224, and the slider element 226. At least one of the actuator side ferromagnetic block 216 and the medical device side ferromagnetic block 224 includes a magnet 214, and the actuator is coupled to the medical device by a magnetic coupling force. The actuator will decouple from the medical device when a decoupling force is greater than a predetermined amount.

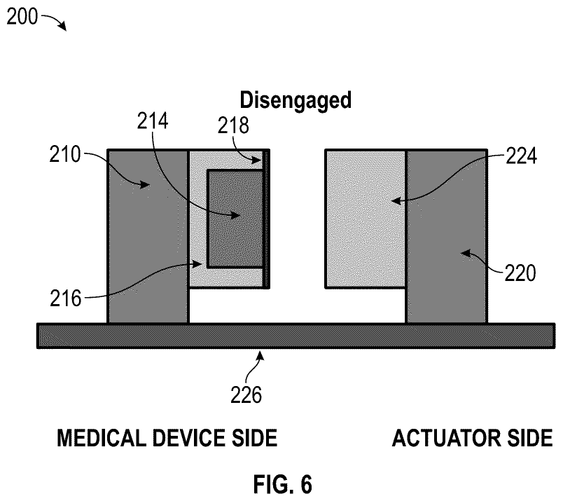

FIG. 5 illustrates a slider 126 that is a slider mechanism supporting some features of the breakaway connection 200 of FIG. 4 in an engaged orientation. FIG. 6 illustrates the breakaway connection 200 of FIG. 5 in a disengaged orientation.

The slider 226 of FIGS. 5 and 6 is an exemplary linear motion device configured as a slide mechanism for implementing a breakaway connection according to an exemplary embodiment. The breakaway apparatus 200 interconnects the medical device 230 on the medical device side with the actuator 240 configured as a motor, drive, motorized drive assembly, or the like on the actuator side. The slider 226 is a linear guide rail and can have an aluminum pusher on the medical device side and an aluminum pusher on the actuator side. The magnet 214 can be, for example, an N52 NdFeB magnet or other type of magnet. Linear guide rails, guideways, and slides are mechanical systems composed of rails and bearings that support and move physical loads along a linear path with a low coefficient of friction. These are generally considered as rolling element or plane bushing types. Common types of guideways and bearings include profiled (square) rails with recirculating ball bearing blocks, guideways for roller bearings, and round rails with recirculating ball bushings or plane bushings. The slides can be saddle, cantilever, or other types of slides. The slider 226 can have a plurality of guide rails laid in parallel and straight at a predetermined distance. A plurality of sliders can be arranged on each of the plurality of guide rails and movable on the guide rails, and a movable part that is bridged over all of the guide rails and is supported by the plurality of sliders and movable along the plurality of guide rails. See FIG. 19 for more details about slide mechanisms according to some embodiments.

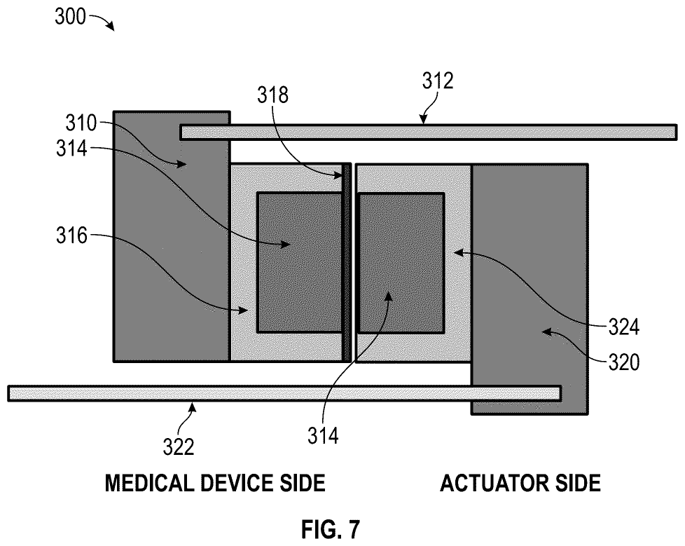

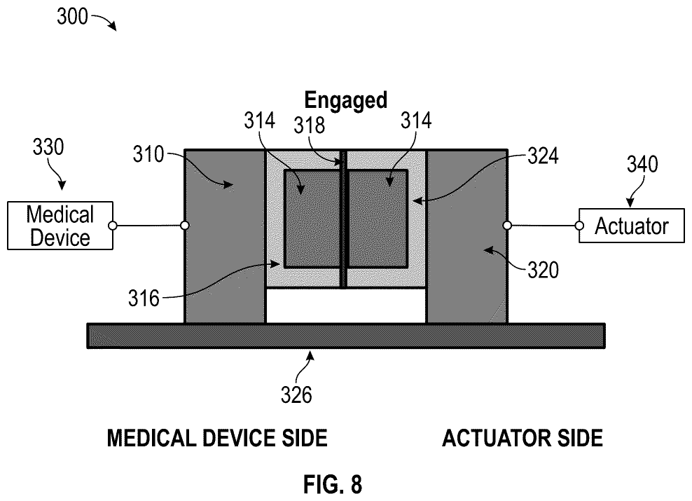

FIG. 7 shows a breakaway apparatus 300 with two magnets and a spacer according to an exemplary embodiment. The breakaway apparatus 300 interconnects a medical or treatment device 330 on the medical device side with an actuator 340 configured as a motor, drive, motorized drive assembly, or the like on the actuator side (see FIG. 8). The drive assembly is releasably connected to the medical device and disconnects from the medical device in response to a coupling force that is greater than or equal to a desired or predetermined amount. The drive assembly is configured as a rotational drive assembly to impart rotational movement to at least one drive wire of the medical device. The drive wire can be a monolithic material wire configured for both push and pull motions, and can include one or more drive wires, push-pull wires, support wires, tendons, strands or other types of wires, and the drive assembly can drive bendable sections of the medical device by pushing and/or pulling the driving wires in a push/pull or insertion/pulling-out direction. The wire(s) are preferably made out of a single material and not a braid or coil. The medical device is preferably a flexible device and can be a catheter, endoscope, colonoscope, bronchoscope, ablation device, or other type of medical device. The breakaway connection 300 is similar to FIG. 4 but also includes an additional magnet 314 on the treatment side.

The actuator side includes one or more of an actuator support 310, an actuator pusher 312, a magnet 314, a ferromagnetic block 316, and can include other elements or components. The actuator support 310 and actuator pusher 312 can be configured together as the same entity according to some embodiments. The treatment side includes one or more of a treatment support 320, a treatment wire pusher 322, a ferromagnetic block 324, and an additional magnet 314, and can include other elements or components. The magnets 314 can be contained within the each of the ferromagnetic blocks 316, 324 and have a coupling force capability that depends on characteristics including the material, shape, configuration, weight, temperature, composition, workload mass, and can include a variety of other factors.

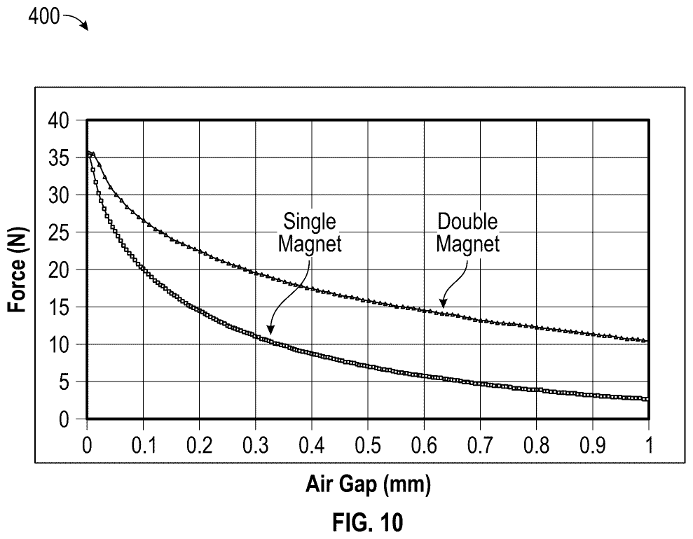

The breakaway connection 300 includes two magnets 314 instead of one as in FIGS. 1 and 4. It is important that both polarities of the magnets 314 are facing the same direction to create an attracting force. The advantage of two magnets 314 as opposed to one is it allows for not only higher coupling force but also less deviation in force per change in gap. The advantages of two magnets 314 instead of one is demonstrated in the diagram 400 of FIG. 10 showing single versus double magnet force results in a pull test data of a single magnet versus a double magnet.

The actuator pusher 312 exerts linear force that is provided by a motor or motorized drive assembly. The force acts through the breakaway connection 300 and exerts itself onto the treatment wire pusher 322. The actuator support 310 and the treatment support 320 present on both sides guide the force from each respective pusher 312, 322 to the breakaway components. The actuator support 110 and the treatment support 320 are preferably rigid to handle forces and can be made out of non-ferrous metal such as aluminum or the like. The medical device generally has a small pitch circle for drive wires so the magnetic force should not exceed a predetermined level large enough to fit axially among the wires without interfering with each other. The breakaway is offset and that is why the supports 310, 320 exist for the actuator pusher side and for the treatment side. The ferromagnetic blocks 316, 324 are made of strong metal material having predetermined strength criteria, e.g. tensile strength, compressive strength, yield strength, impact strength, etc., such as steel, steel alloys, tungsten, titanium, chromium, or the like, e.g. 1018 steel, etc., and serve to permeate magnetic flux and create a bond between the two sides, the actuator support 310 and the treatment support 320, out of magnetic force that is characterized as being less than a predetermined or desired coupling force.

The breakaway connection 300 can have a compressive breakaway mechanism located in the actuator side or proximal end of the medical device that decouples the actuator from the drive wires once a threshold force is passed or has occurred. This breakaway mechanism is a passive mechanical assembly that does not rely on electrical power to operate. Specifically, the breakaway mechanism 300 with the magnet 314 contained within the ferromagnetic block 316 can decouple the actuator from the drive wire once the threshold force is passed.

A breakaway apparatus according to some embodiments can include an actuator, a medical device, and the breakaway connection 300 coupling the actuator and the medical device, the breakaway connection 300 having an actuator portion on the actuator and a medical device portion on the medical device. The actuator portion can include the actuator support 310, the actuator side ferromagnetic block 316, and the slider element 326. The medical device portion can include the medical device support 120, the medical device side ferromagnetic block 324, and the slider element 126. At least one of the actuator side ferromagnetic block 316 and the medical device side ferromagnetic block 324 includes a magnet 314, and the actuator is coupled to the medical device by a magnetic coupling force. The actuator will decouple from the medical device when a decoupling force is greater than a predetermined amount.

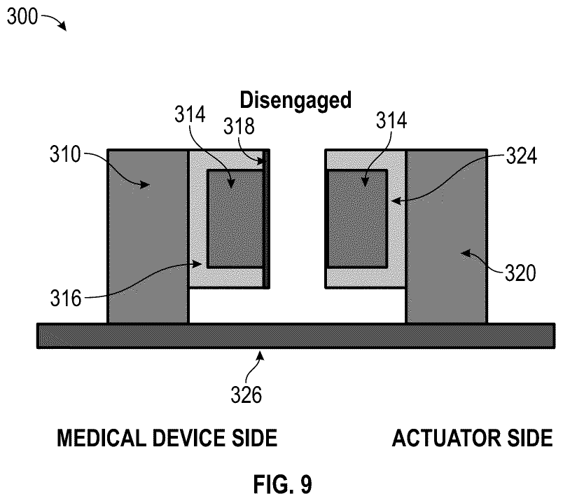

FIG. 8 illustrates a slider 326 that is a slider mechanism supporting some features of the breakaway connection 300 of FIG. 7 in an engaged orientation. FIG. 9 illustrates the breakaway connection 300 of FIG. 8 in a disengaged orientation.

The slider 326 of FIGS. 8 and 9 is an exemplary linear motion device configured as a slide mechanism for implementing a breakaway connection according to an exemplary embodiment. The slider 326 is a linear guide rail and can have an aluminum pusher on the medical device side and an aluminum pusher on the actuator side. The magnets 314 can be, for example, an N52 NdFeB magnet or other type of magnet. Linear guide rails, guideways, and slides are mechanical systems composed of rails and bearings that support and move physical loads along a linear path with a low coefficient of friction. These are generally considered as rolling element or plane bushing types. Common types of guideways and bearings include profiled (square) rails with recirculating ball bearing blocks, guideways for roller bearings, and round rails with recirculating ball bushings or plane bushings. The slides can be saddle, cantilever, or other types of slides. The slider 226 can have a plurality of guide rails laid in parallel and straight at a predetermined distance. A plurality of sliders can be arranged on each of the plurality of guide rails and movable on the guide rails, and a movable part that is bridged over all of the guide rails and is supported by the plurality of sliders and movable along the plurality of guide rails. See FIG. 19 for more details about slide mechanisms according to some embodiments.

The drive wires preferably have elastic or super-elastic characteristics or attributes to act as a safety mechanism and can be made from suitable materials including nickel titanium alloy (Nitinol), Flexinoff, nickel-chromium, copper, aluminum, stainless steel, silver, gold, polycarbonate, polypropylene, polyurethane, silicone, and can include other metallic, non-metallic, or polymer materials, or combinations thereof. Super-elastic materials, such as Ntinol, have a flexibility 10-20 times greater than stainless steel and can handle significant bending without buckling, kinking or permanent deformation. Nitinol is a non-ferromagnetic super-elastic shape memory alloy with a lower magnetic susceptibility than stainless steel, and provides clear images with less artifacts than stainless steel. Super-elastic materials are beneficial to medical applications by enhancing performance in kink resistance, fatigue resistance, elastic deployment, thermal deployment, biocompatibility, etc.

A tensile breakaway mechanism can be located in the medical device. The tensile breakaway mechanism can be configured with super-elastic drive wires, such as Nitinol. At a force equal to or greater than a predetermined threshold force, super-elastic drive wires of the breakaway mechanism can allow for a large stroke without increasing the pull force. The tensile breakaway mechanism can also be configured so that the breakaway mechanism will break in a safe manner at a weakest point when a force occurs equal to or greater than the predetermined threshold force.

A key feature of the breakaway mechanism according to some embodiments is to locate a dedicated breakaway mechanism for tensile and compression forces at various locations. Specifically, the compression breakaway mechanism can be located at the actuator while the tensile breakaway mechanism can be located at the steerable medical device.

The failures with compression force on the wires can potentially happen in any area through push-pull wires, connection parts between the wires, and the actuator. These failures can include buckling of the wires and anchor failures. Therefore, a preferable configuration to prevent these types of failures would be at the most proximal location during the force transmission line with the push-pull wires.

On the other hand, the failures with tensile forces can happen mainly at an anchor part or at the thin diameter part of the push-pull wires, i.e., in the medical device body. The breakaway structure for the tensile force can be located at a more distal part than the actuator to cover the failures of the tensile forces. By locating the tensile breakaway mechanism at the medical device, the actuator and connection part can be simplified and more space can be provided for the compression breakaway mechanism to minimize the size of the breakaway connection 300.

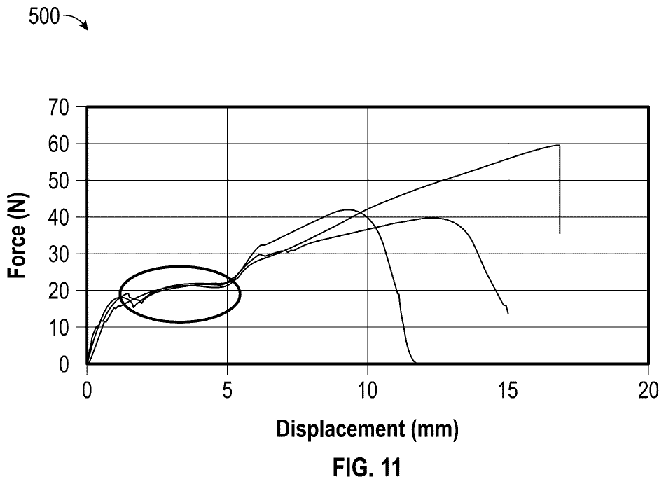

In a case where the tensile breakaway mechanism is located in the medical device and configured with super-elastic drive wires, such as Nitinol, the super-elastic property of the Nitinol wire can act as a safety mechanism. As shown in the tensile test diagram 500 of FIG. 11 (NiTi Wire Tensile Test, 0.25 mm/s, n=3), once a force threshold is passed, 20 N (newtons) for example, the force/displacement ratio can transition from a linear region to a super-elastic plateau region (see circled section) where increased stroke induces no force increase. This breakaway mechanism can only work in the pull (tensile) direction as the wire will prolapse in compression before entering the super-elastic region. In this case, the super-elastic property of the Nitinol wire can act as a safety mechanism where a force equal to or greater than a predetermined force can allow for a large stroke without increasing the pull force. Super-elastic materials reversibly deform to a high strain in response to high stress.

In a case where the tensile breakaway mechanism is configured so that the breakaway mechanism will break in a safe manner at a weakest point when a force occurs that is equal to or greater than the predetermined threshold force, the tensile breakaway mechanism can include an ultimate failure mechanism that acts as a backup to the previous breakaway mechanism configured with super-elastic Nitinol drive wires. This can be achieved by having a concentrated failure point located in the proximal section of the medical device. In the prior state, ultimate failure occurs in the medical device tip, which is potentially harmful to the patient. In this case, failure always occurs in the hub of the medical device.

This can be achieved by having an unsupported length configured to prolapse at a force less than or equal to the minimum prolapse force in the medical device.

Electrical sensing and software intervention can be implemented with a force sensor by locating or placing the force sensor between the actuator and the drive wire according to some embodiments. The force sensor can detect force in any direction or along each coordinate axis in an XYZ three-dimensional coordinate system. When a force is induced between the actuator and the drive wire, the force sensor can output a voltage to a controller. With programmed software or firmware limits, when a voltage is detected or sensed that becomes higher, passes, or exceeds a desired or predetermined threshold voltage, the controller can stop motion and send a warning signal to the user. This can be referred to as a “motor stop circuit”.

This type of threshold voltage can occur at a lower force than the mechanical breakaway mechanism as it is easier to recover to a normal operational state since no damage or mechanism engagement has occurred. While this electrical sensing can prevent the actuator from inducing more force into the breakaway mechanism, outside factors can still cause additional force to be induced into the medical device. This is when the mechanical threshold takes effect.

The force sensor can also trigger an “auto-relax mode” which can occur before the “motor stop circuit”, but at an upper limit of acceptable operating wire forces. This can be a software algorithm that pauses operation and relieves tension/compression in the wire for better performance and to help prevent higher wire forces from being induced.

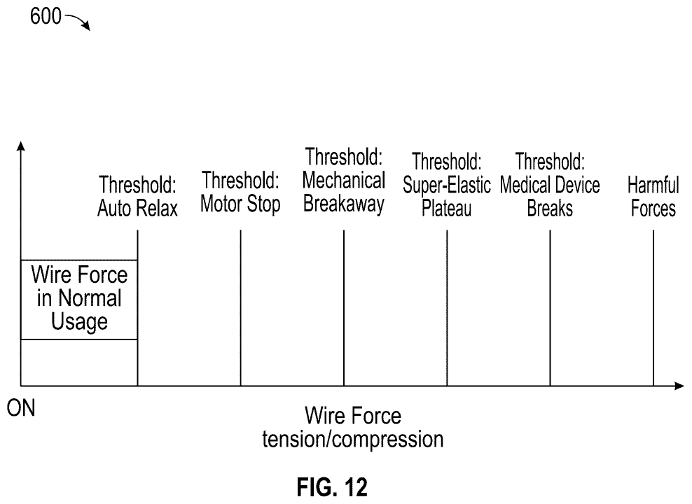

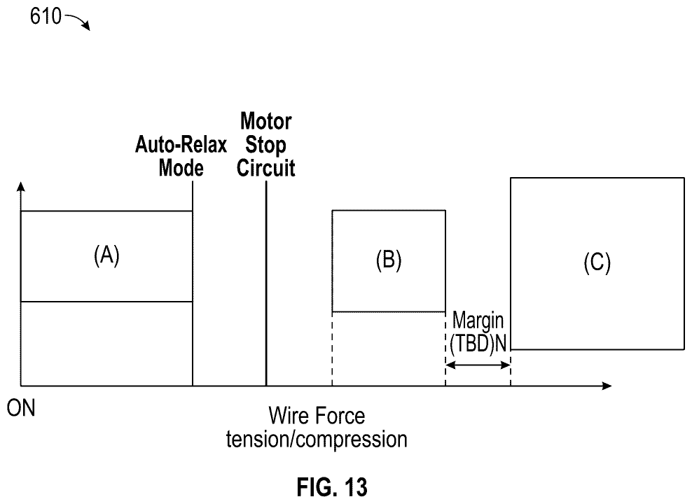

Force management for different force thresholds can be implemented through user or software intervention according to some embodiments. As illustrated in the force management diagram 600 of FIG. 12 and force management diagram of FIG. 13, force thresholds for force management purposes can include an auto-relax threshold, a motor stop threshold, a mechanical breakaway threshold, a super-elastic plateau threshold, a treatment device breaks threshold, a harmful forces threshold, and can include other type of force thresholds. FIG. 13 shows a force management diagram 610, where a wire force tension/compression range through an auto-relax mode can occur in section (A). A wire force at breakaway can occur in section (B), and wire tension that affects an object, such as the human body, or wire tension at which the medical device is broken can occur in section (C). Wire tension can be in use in section (A) up to an auto-relax mode. A margin between second (B) and section (C) is a wire force tension/compression range that is unknown and is to be determined during operation.

The processor, controller, user, or combinations thereof can execute force management software, whereby force sensor readings can be periodically detected. The auto-relax mode or threshold can occur at an upper limit of wire forces during normal use. The motor stop threshold can stop all actuator motor or motorized drive assembly motion and can prevent any additional forces from being induced by the actuator to the medical device. The mechanical breakaway threshold is a passive mechanical breakaway threshold, i.e., magnetic breakaway, which engages regardless of electrical signals. The super-elastic plateau threshold is the super-elastic threshold of the Nitinol driving wire, which only occurs in tension, and prevents additional force from being induced into the medical device tip. The medical device breaks threshold occurs in the proximal section of the medical device. Due to the multiplicity of safety thresholds, the chances of harmful forces being induced in the medical device tip are highly unlikely.

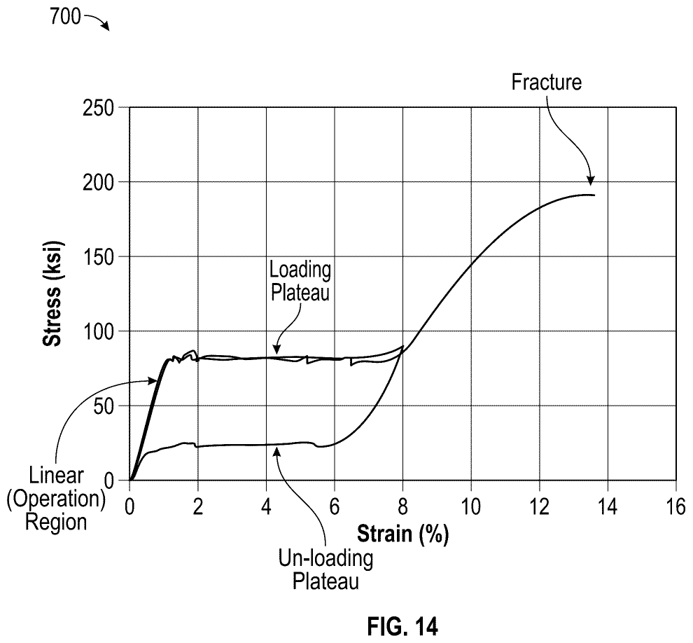

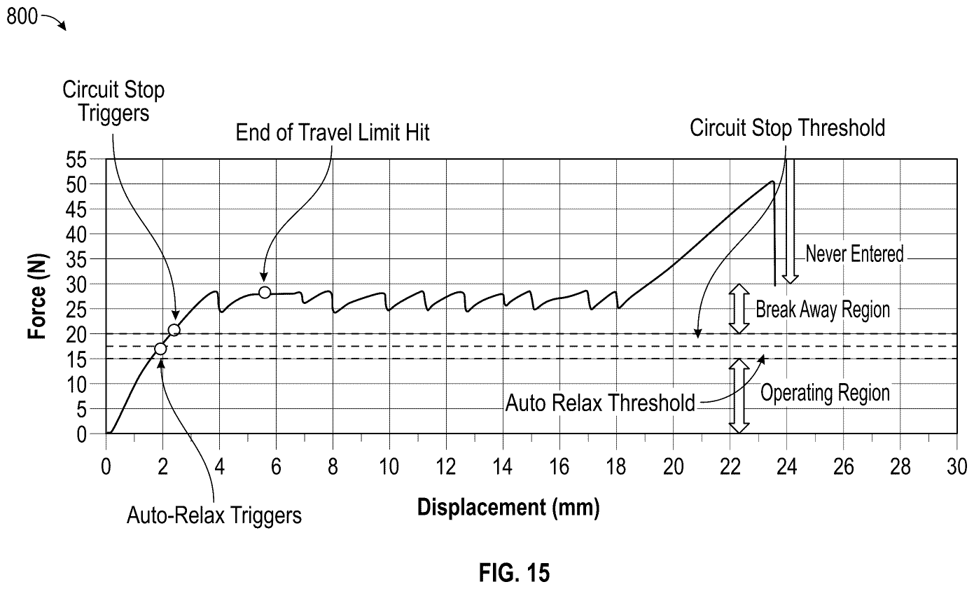

FIG. 14 illustrates a stress strain curve 700 for super-elastic Nitinol (0.013″ Niti #1 USN®). FIG. 15 illustrates a force displacement curve 800 from tested Nitinol wire.

Some material, such as Nitinol wire, do not follow a standard stress/strain curve but instead exhibit a super-elastic material behavior. Nitinol has advantageous qualities regarding strain-resistance and shape memory. Nitinol is able to remember its shape and return to its original shape when heated. With standard materials, there is an elastic linear region where stress/strain is a constant and a material will return to its original shape after de-loading. After a certain strain input, the material enters the plastic region, where stress/strain becomes non-linear, and the material no longer returns to its original shape. With super-elastic Nitinol and the stress strain curve 700 shown in FIG. 14, at strain<˜1% the material behaves like a standard material with linear stress/strain. However, when strain is >˜1%, it enters the super-elastic region where additional strain result in no additional stress (see the loading plateau). When the Nitinol is de-loaded, it will have a hysteresis shown in the un-loading plateau, however it ultimately returns to its original shape with no plastic deformation.

Given these properties of super elastic Nitinol, by incorporating super-elastic material such as Nitinol in the breakaway configurations according to the present disclosure, the material functions as a breakaway for tensile forces. As shown in the force displacement curve 800 of a crimpled pusher rod to drive wire tensile test in FIG. 15 from tested snake Nitinol wire, given the diameter of the Nitinol is optimized for the performance of a snake catheter, it is able to achieve a pull force of ˜25 N before entering the super-elastic region. Once the region is entered the drive wire can no longer induce higher forces unless ˜8% strain is obtained. However, the actuator limits this from happening through end of travel limits and hard stops.

The actuator contains an end of travel limit sensor and hard stop preventing the catheter wire from ever reaching 8% strain where is exits the super-elastic region and induces forces>30N. The electronic limit sensor can be located at roughly 14 mm from center of travel while the hard stop is located roughly at 15 mm of travel.

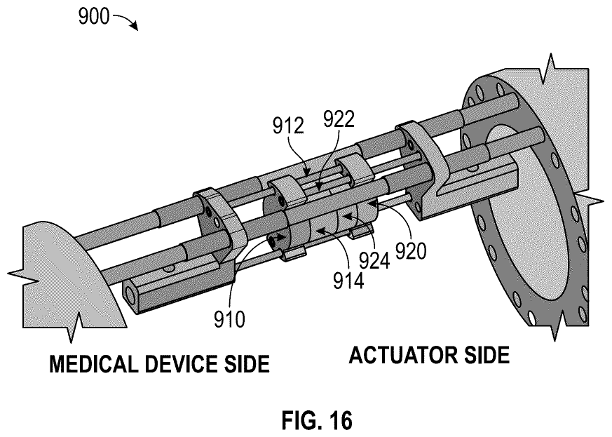

FIG. 16 shows a breakaway connection 900 according to an exemplary embodiment that includes an actuator side and a medical device or treatment side. The breakaway connection 900 interconnects a medical or treatment device on the medical device side with an actuator configured as a motor, drive, motorized drive assembly, or the like on the actuator side (not shown, see FIG. 2). The actuator side includes one or more of a support 910, an actuator pusher 912, a magnet (not shown), a ferromagnetic block 914, and can include other elements or components. The treatment side includes one or more of a support 920, a treatment wire pusher 922, a ferromagnetic block 924, and can include other elements or components. The magnet is contained within the ferromagnetic block 914 on the actuator side and has a coupling force capability that depends on characteristics including the material, shape, configuration, weight, temperature, composition, workload mass, and can include a variety of other factors.

The actuator pusher 912 exerts linear force that is provided by a motor, a motorized drive assembly, or the like. The force acts through the breakaway connection 900 exerting itself onto the treatment wire pusher 922. The actuator support 910 and the treatment support 920 present on both sides guide the force from each respective pusher 912, 922 to the breakaway components. The supports 910, 920 are preferably rigid to handle forces and can be made out of non-ferrous metal such as aluminum or the like. The medical device generally has a small pitch circle for drive wires so the magnetic force should not exceed a predetermined level large enough to fit axially among the wires without interfering with each other. The breakaway is offset and that is why the supports 910, 920 exist for the actuator pusher side and for the treatment side. The ferromagnetic blocks 914, 924 are made of strong metal material, such as steel or the like, e.g. 1018 steel, etc., and serve to permeate magnetic flux and create a bond between the two sides, the actuator support 910 and the treatment support 920, out of magnetic force that is characterized as being equal to a predetermined or desired coupling force.

The breakaway connection 900 can have a compressive breakaway mechanism located in the actuator side or proximal end of the medical device that decouples the actuator from the drive wire or drive wire once a threshold force is passed or has occurred. This breakaway mechanism is a passive mechanical assembly that does not rely on electrical power to operate. Specifically, the breakaway mechanism 900 with the magnet contained within the ferromagnetic block 914 can decouple the actuator from the drive wire once the threshold force is passed.

The drive wires preferably have elastic or super-elastic characteristics or attributes to act as a safety mechanism and can be made from suitable materials including Nitinol, Flexinoff, nickel-chromium, copper, aluminum, stainless steel, silver, gold, polycarbonate, polypropylene, polyurethane, silicone, and can include other metallic, non-metallic, or polymer materials, or combinations thereof. Super-elastic materials, such as Nitinol, have a flexibility 10-20 times greater than stainless steel and can handle significant bending without buckling, kinking or permanent deformation. Nitinol is a non-ferromagnetic super-elastic shape memory alloy with a lower magnetic susceptibility than stainless steel, and provides clear images with less artifacts than stainless steel. Super-elastic materials are beneficial to medical applications by enhancing performance in kink resistance, fatigue resistance, elastic deployment, thermal deployment, biocompatibility, etc.

A tensile breakaway mechanism can be located in the medical device. The tensile breakaway mechanism can be configured with super-elastic drive wires, such as Nitinol. At a force equal to or greater than a predetermined threshold force, super-elastic drive wires of the breakaway mechanism can allow for a large stroke without increasing the pull force. The tensile breakaway mechanism can also be configured so that the breakaway mechanism will break in a safe manner at a weakest point when a force occurs equal to or greater than the predetermined threshold force.

A key feature of the breakaway mechanism according to some embodiments is to locate a dedicated breakaway mechanism for tensile and compression forces at various locations. Specifically, the compression breakaway mechanism can be located at the actuator while the tensile breakaway mechanism can be located at a steerable medical device.

The failures with compression force on the wires can potentially happen in any area through push-pull wires, connection parts between the wires, and the actuator. These failures can include buckling of the wires and anchor failures. Therefore, a preferable configuration to prevent these types of failures would be at the most proximal location during the force transmission line with the push-pull wires.

On the other hand, the failures with tensile forces can happen mainly at an anchor part or at the thin diameter part of the push-pull wires, i.e., in the medical device body. The breakaway structure for the tensile force can be located at a more distal part than the actuator to cover the failures of the tensile forces. By locating the tensile breakaway mechanism at the medical device, the actuator and connection part can be simplified and more space can be provided for the compression breakaway mechanism to minimize the size of the breakaway connection 900.

In a case where the tensile breakaway mechanism is located in the medical device and configured with super-elastic drive wires, such as Nitinol, the super-elastic property of the Nitinol wire can act as a safety mechanism. As shown in the tensile test diagram 500 of FIG. 11, once a force threshold is passed, 20 N (newtons) for example, the force/displacement ratio can transition from a linear region to a super-elastic plateau region (see circled section) where increased stroke induces no force increase. This breakaway mechanism can only work in the pull (tensile) direction as the wire will prolapse in compression before entering the super-elastic region. In this case, the super-elastic property of the Nitinol wire can act as a safety mechanism where a force equal to or greater than a predetermined force can allow for a large stroke without increasing the pull force.

In a case where the tensile breakaway mechanism is configured so that the breakaway mechanism will break in a safe manner at a weakest point when a force occurs that is equal to or greater than the predetermined threshold force, the tensile breakaway mechanism can include an ultimate failure mechanism that acts as a backup to the previous breakaway mechanism configured with super-elastic Nitinol drive wires. This can be achieved by having a concentrated failure point located in the proximal section of the medical device. In the prior state, ultimate failure occurs in the medical device tip, which is potentially harmful to the patient. In this case, failure always occurs in the hub of the medical device.

This can be achieved by having an unsupported length configured to prolapse at a force less than or equal to the minimum prolapse force in the medical device.

A force sensor can be located between the actuator and the drive wire according to some embodiments. When a force is induced between the actuator and the drive wire, the sensor can output a voltage to a controller. With programmed software or firmware limits, when a threshold voltage is passed, the controller can stop motion and send a warning signal to the user. This can be referred to as a “motor stop circuit”.

This type of threshold voltage can occur at a lower force than the mechanical breakaway mechanism as it is easier to recover to a normal operational state since no damage or mechanism engagement has occurred. While this electrical sensing can prevent the actuator from inducing more force into the breakaway mechanism, outside factors can still cause additional force to be induced into the medical device. This is when the mechanical threshold takes effect.

The force sensor can also trigger an “auto-relax mode” which can occur before the “motor stop circuit”, but at an upper limit of acceptable operating wire forces. This can be a software algorithm that pauses operation and relieves tension/compression in the wire for better performance and to help prevent higher wire forces from being induced.

Force management for different force thresholds can be implemented according to some embodiments. As illustrated in the force management diagram 600 of FIG. 12, force thresholds for force management purposes can include an auto-relax threshold, a motor stop threshold, a mechanical breakaway threshold, a super-elastic plateau threshold, a medical device breaks threshold, a harmful force threshold, and can include other types of force thresholds.

The auto-relax mode or threshold can occur at an upper limit of wire forces during normal use. The motor stop threshold can stop all actuator motion and can prevent any additional forces from being induced by the actuator to the medical device. The mechanical breakaway threshold is a passive mechanical breakaway threshold, i.e., magnetic breakaway, which engages regardless of electrical signals. The super-elastic plateau threshold is the super-elastic threshold of the Nitinol driving wire, which only occurs in tension, and prevents additional force from being induced into the medical device tip. The medical device breaks threshold occurs in the proximal section of the medical device. Due to the multiplicity of safety thresholds, the chances of harmful forces being induced in the medical device tip are highly unlikely.

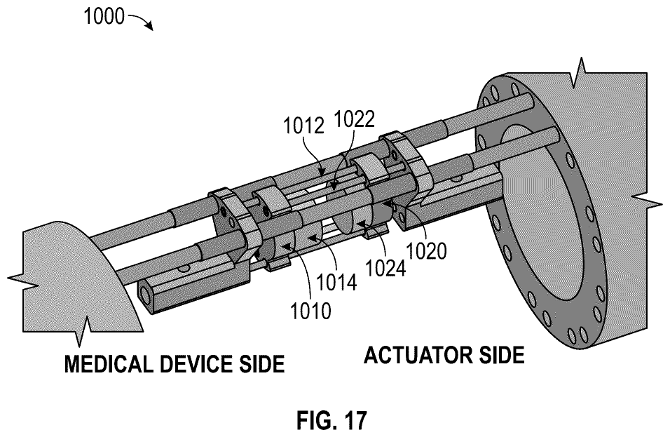

FIG. 17 shows a breakaway connection 1000 according to an exemplary embodiment that includes an actuator side and a medical device or treatment side. The breakaway connection 1000 interconnects a medical or treatment device on the medical device side with an actuator configured as a motor, drive, motorized drive assembly, or the like on the actuator side (not shown, see FIG. 2). The actuator side includes one or more of a support 1010, an actuator pusher 1012, a magnet (not shown), a ferromagnetic block 1014, and can include other elements or components. The actuator support 1010 and actuator pusher 1012 can be configured together as the same entity according to some embodiments. The treatment side includes one or more of a support 1020, a treatment wire pusher 1022, a ferromagnetic block 1024, and can include other elements or components. The magnet is contained within the ferromagnetic block 1014 on the actuator side and has a coupling force capability that depends on characteristics including the material, shape, configuration, weight, temperature, composition, workload mass, and can include a variety of other factors.

The actuator pusher 1012 exerts linear force that is provided by a motor or motorized drive assembly. The force acts through the breakaway connection 1000 exerting itself onto the treatment wire pusher 1022. The actuator support 1010 and the treatment support 1020 present on both sides guide the force from each respective pusher 1012, 1022 to the breakaway components. The supports 1010, 1020 are preferably rigid to handle forces and can be made out of non-ferrous metal such as aluminum or the like. The medical device generally has a small pitch circle for drive wires so the magnetic force should not exceed a predetermined level large enough to fit axially among the wires without interfering with each other. The breakaway is offset and that is why the supports 1010, 1020 exist for the actuator pusher side and the for the treatment side. The ferromagnetic blocks 1014, 1024 are made of strong metal material, such as steel or the like, e.g. 1018 steel, etc., and serve to permeate magnetic flux and create a bond between the two sides, the actuator support 1010 and the treatment support 1020, out of magnetic force that is characterized as being equal to a predetermined or desired coupling force.

The breakaway connection 1000 can have a compressive breakaway mechanism located in the actuator side or proximal end of the medical device that decouples the actuator from the drive wire or guidewire once a threshold force is passed or has occurred. This breakaway mechanism is a passive mechanical assembly that does not rely on electrical power to operate. Specifically, the breakaway mechanism with the magnet contained within the ferromagnetic block 1016 can decouple the actuator from the drive wire once the threshold force is passed.

The drive wires preferably have elastic or super-elastic characteristics or attributes to act as a safety mechanism and can be made from suitable materials including Nitinol, Flexinoff, nickel-chromium, copper, aluminum, stainless steel, silver, gold, polycarbonate, polypropylene, polyurethane, silicone, and can include other metallic, non-metallic, or polymer materials, or combinations thereof. Super-elastic materials, such as Nitinol, have a flexibility 10-20 times greater than stainless steel and can handle significant bending without buckling, kinking or permanent deformation. Nitinol is a non-ferromagnetic super-elastic shape memory alloy with a lower magnetic susceptibility than stainless steel, and provides clear images with less artifacts than stainless steel. Super-elastic materials are beneficial to medical applications by enhancing performance in kink resistance, fatigue resistance, elastic deployment, thermal deployment, biocompatibility, etc.

A tensile breakaway mechanism can be located in the medical device. The tensile breakaway mechanism can be configured with super-elastic drive wires, such as Nitinol. At a force equal to or greater than a predetermined threshold force, super-elastic drive wires of the breakaway mechanism can allow for a large stroke without increasing the pull force. The tensile breakaway mechanism can also be configured so that the breakaway mechanism will break in a safe manner at a weakest point when a force occurs equal to or greater than the predetermined threshold force.

A key feature of the breakaway mechanism according to some embodiments is to locate a dedicated breakaway mechanism for tensile and compression forces at various locations. Specifically, the compression breakaway mechanism can be located at the actuator while the tensile breakaway mechanism can be located at the steerable medical device.

The failures with compression force on the wires can potentially happen in any area through push-pull wires, connection parts between the wires, and the actuator. These failures can include buckling of the wires and anchor failures. Therefore, a preferable configuration to prevent these types of failures would be at the most proximal location during the force transmission line with the push-pull wires.

On the other hand, the failures with tensile forces can happen mainly at an anchor part or at the thin diameter part of the push-pull wires, i.e., in the medical device body. The breakaway structure for the tensile force can be located at a more distal part than the actuator to cover the failures of the tensile forces. By locating the tensile breakaway mechanism at the medical device, the actuator and connection part can be simplified and more space can be provided for the compression breakaway mechanism to minimize the size of the breakaway connection 100.

In a case where the tensile breakaway mechanism is located in the medical device and configured with super-elastic drive wires, such as Nitinol, the super-elastic property of the Nitinol wire can act as a safety mechanism. As shown in the tensile test diagram 500 of FIG. 11, once a force threshold is passed, 20 N (newtons) for example, the force/displacement ratio can transition from a linear region to a super-elastic plateau region (see circled section) where increased stroke induces no force increase. This breakaway mechanism can only work in the pull (tensile) direction as the wire will prolapse in compression before entering the super-elastic region. In this case, the super-elastic property of the Nitinol wire can act as a safety mechanism where a force equal to or greater than a predetermined force can allow for a large stroke without increasing the pull force.

In a case where the tensile breakaway mechanism is configured so that the breakaway mechanism will break in a safe manner at a weakest point when a force occurs that is equal to or greater than the predetermined threshold force, the tensile breakaway mechanism can include an ultimate failure mechanism that acts as a backup to the previous breakaway mechanism configured with super-elastic Nitinol drive wires. This can be achieved by having a concentrated failure point located in the proximal section of the medical device. In the prior state, ultimate failure occurs in the medical device tip, which is potentially harmful to the patient. In this case, failure always occurs in the hub of the medical device.

This can be achieved by having an unsupported length configured to prolapse at a force less than or equal to the minimum prolapse force in the medical device.

A force sensor can be located between the actuator and the drive wire according to some embodiments. When a force is induced between the actuator and the drive wire, the sensor can output a voltage to a controller. With programmed software or firmware limits, when a threshold voltage is passed, the controller can stop motion and send a warning signal to the user. This can be referred to as a “motor stop circuit”.

This type of threshold voltage can occur at a lower force than the mechanical breakaway mechanism as it is easier to recover to a normal operational state since no damage or mechanism engagement has occurred. While this electrical sensing can prevent the actuator from inducing more force into the breakaway mechanism, outside factors can still cause additional force to be induced into the medical device. This is when the mechanical threshold takes effect.

The force sensor can also trigger an “auto-relax mode” which can occur before the “motor stop circuit”, but at an upper limit of acceptable operating wire forces. This can be a software algorithm that pauses operation and relieves tension/compression in the wire for better performance and to help prevent higher wire forces from being induced.

Force management for different force thresholds can be implemented according to some embodiments. As illustrated in the force management diagram 600 of FIG. 12, force thresholds for force management purposes can include an auto-relax threshold, a motor stop threshold, a mechanical breakaway threshold, a super-elastic plateau threshold, a medical device break threshold, a harmful force threshold, and can include other type of force thresholds.

The auto-relax mode or threshold can occur at an upper limit of wire forces during normal use. The motor stop threshold can stop all actuator motion and can prevent any additional forces from being induced by the actuator to the medical device. The mechanical breakaway threshold is a passive mechanical breakaway threshold, i.e. magnetic breakaway, which engages regardless of electrical signals. The super-elastic plateau threshold is the super-elastic threshold of the Nitinol driving wire, which only occurs in tension, and prevents additional force from being induced into the medical device tip. The medical device breaks threshold occurs in the proximal section of the medical device. Due to the multiplicity of safety thresholds, the chances of harmful forces being induced in the medical device tip are highly unlikely.

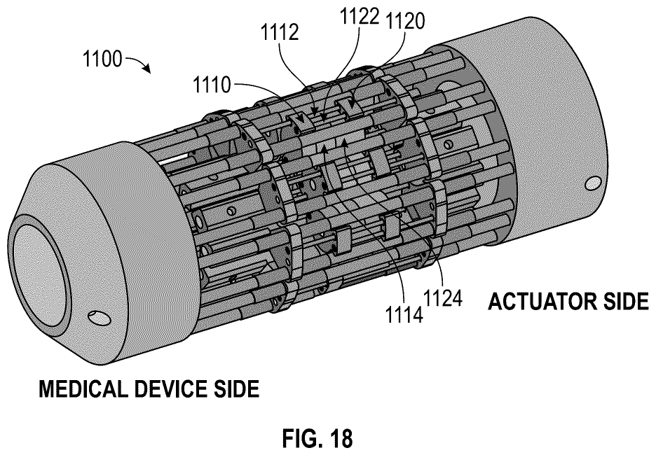

FIG. 18 shows a breakaway connection 1100 according to an exemplary embodiment that includes an actuator side and a medical device or treatment side. The breakaway connection 1100 interconnects a medical or treatment device on the medical device side with an actuator configured as a motor, drive, motorized drive assembly, or the like on the actuator side (not shown, see FIG. 2). The breakaway connection 1100 includes a plurality of breakaway connections similar to the breakaway connection 900 shown in FIG. 16. The actuator side includes one or more of a support 1110, an actuator pusher 1112, a magnet (not shown), a ferromagnetic block 1114, and can include other elements or components. The actuator support 1110 and actuator pusher 1112 can be configured together as the same entity according to some embodiments. The treatment side includes one or more of a support 1120, a treatment wire pusher 1122, a ferromagnetic block 1124, and can include other elements or components. The magnet is contained within the ferromagnetic block 1114 on the actuator side and has a coupling force capability that depends on characteristics including the material, shape, configuration, weight, temperature, composition, workload mass, and can include a variety of other factors.

The actuator pusher 1112 exerts linear force that is provided by a motor or motorized drive assembly. The force acts through the breakaway connection 1100 exerting itself onto the treatment wire pusher 1122. The actuator support 1110 and the treatment support 1120 present on both sides guide the force from each respective pusher 1112, 1122 to the breakaway components. The supports 1110, 1120 are preferably rigid to handle forces and can be made out of non-ferrous metal such as aluminum or the like. The medical device generally has a small pitch circle for drive wires so the magnetic force should not exceed a predetermined level large enough to fit axially among the wires without interfering with each other. The breakaway is offset and that is why the supports 1110, 1120 exist for the actuator pusher side and the for the medical device side. The ferromagnetic blocks 1114, 1124 are made of strong metal material, such as steel or the like, e.g. 1018 steel, etc., and serve to permeate magnetic flux and create a bond between the two sides, the actuator support 1110 and the treatment support 1120, out of magnetic force that is characterized as being equal to a predetermined or desired coupling force.

The breakaway connection 1100 can have a compressive breakaway mechanism located in the actuator side or proximal end of the treatment that decouples the actuator from the drive wire once a threshold force is passed or has occurred. This breakaway mechanism is a passive mechanical assembly that does not rely on electrical power to operate. Specifically, the breakaway mechanism with the magnet contained within the ferromagnetic block 1116 can decouple the actuator from the drive wire once the threshold force is passed.

The drive wires preferably have elastic or super-elastic characteristics or attributes to act as a safety mechanism and can be made from suitable materials including Nitinol, Flexinoff, nickel-chromium, copper, aluminum, stainless steel, silver, gold, polycarbonate, polypropylene, polyurethane, silicone, and can include other metallic, non-metallic, or polymer materials, or combinations thereof. Super-elastic materials, such as Nitinol, have a flexibility 10-20 times greater than stainless steel and can handle significant bending without buckling, kinking or permanent deformation. Nitinol is a non-ferromagnetic super-elastic shape memory alloy with a lower magnetic susceptibility than stainless steel, and provides clear images with less artifacts than stainless steel. Super-elastic materials are beneficial to medical applications by enhancing performance in kink resistance, fatigue resistance, elastic deployment, thermal deployment, biocompatibility, etc.

A tensile breakaway mechanism can be located in the medical device. The tensile breakaway mechanism can be configured with super-elastic drive wires, such as Nitinol. At a force equal to or greater than a predetermined threshold force, super-elastic drive wires of the breakaway mechanism can allow for a large stroke without increasing the pull force. The tensile breakaway mechanism can also be configured so that the breakaway mechanism will break in a safe manner at a weakest point when a force occurs equal to or greater than the predetermined threshold force.

A key feature of the breakaway mechanism according to some embodiments is to locate a dedicated breakaway mechanism for tensile and compression forces at various locations. Specifically, the compression breakaway mechanism can be located at the actuator while the tensile breakaway mechanism can be located at the steerable medical device.

The failures with compression force on the wires can potentially happen in any area through push-pull wires, connection parts between the wires, and the actuator. These failures can include buckling of the wires and anchor failures. Therefore, a preferable configuration to prevent these types of failures would be at the most proximal location during the force transmission line with the push-pull wires.

On the other hand, the failures with tensile forces generally happen mainly at an anchor part or at the thin diameter part of the push-pull wires, i.e., in the medical device body. The breakaway structure for the tensile force can be located at a more distal part than the actuator to cover the failures of the tensile forces. By locating the tensile breakaway mechanism at the medical device, the actuator and connection part can be simplified and more space can be provided for the compression breakaway mechanism to minimize the size of the breakaway connection 1100.

In a case where the tensile breakaway mechanism is located in the medical device and configured with super-elastic drive wires, such as Nitinol, the super-elastic property of the Nitinol wire can act as a safety mechanism. As shown in the tensile test diagram 500 of FIG. 11, once a force threshold is passed, 20 N (newtons) for example, the force/displacement ratio can transition from a linear region to a super-elastic plateau region (see circled section) where increased stroke induces no force increase. This breakaway mechanism can only work in the pull (tensile) direction as the wire will prolapse in compression before entering the super-elastic region. In this case, the super-elastic property of the Nitinol wire can act as a safety mechanism where a force equal to or greater than a predetermined force can allow for a large stroke without increasing the pull force.

In a case where the tensile breakaway mechanism is configured so that the breakaway mechanism will break in a safe manner at a weakest point when a force occurs that is equal to or greater than the predetermined threshold force, the tensile breakaway mechanism can include an ultimate failure mechanism that acts as a backup to the previous breakaway mechanism configured with super-elastic Nitinol drive wires. This can be achieved by having a concentrated failure point located in the proximal section of the medical device. In the prior state, ultimate failure occurs in the medical device tip, which is potentially harmful to the patient. In this case, failure always occurs in the hub of the medical device.

This can be achieved by having an unsupported length configured to prolapse at a force less than or equal to the minimum prolapse force in the medical device.

A force sensor can be located between the actuator and the drive wire according to some embodiments. When a force is induced between the actuator and the drive wire, the sensor can output a voltage to a controller. With programmed software or firmware limits, when a threshold voltage is passed, the controller can stop motion and send a warning signal to the user. This can be referred to as a “motor stop circuit”.

This type of threshold voltage can occur at a lower force than the mechanical breakaway mechanism as it is easier to recover to a normal operational state since no damage or mechanism engagement has occurred. While this electrical sensing can prevent the actuator from inducing more force into the breakaway mechanism, outside factors can still cause additional force to be induced into the medical device. This is when the mechanical threshold takes effect.

The force sensor can also trigger an “auto-relax mode” which can occur before the “motor stop circuit”, but at an upper limit of acceptable operating wire forces. This can be a software algorithm that pauses operation and relieves tension/compression in the wire for better performance and to help prevent higher wire forces from being induced.

Force management for different force thresholds can be implemented according to some embodiments. As illustrated in the force management diagram 600 of FIG. 12, force thresholds for force management purposes can include an auto-relax threshold, a motor stop threshold, a mechanical breakaway threshold, a super-elastic plateau threshold, a medical device breaks threshold, a harmful force threshold, and can include other type of force thresholds.

The auto-relax mode or threshold can occur at an upper limit of wire forces during normal use. The motor stop threshold can stop all actuator motion and can prevent any additional forces from being induced by the actuator to the medical device. The mechanical breakaway threshold is a passive mechanical breakaway threshold, i.e., magnetic breakaway, which engages regardless of electrical signals. The super-elastic plateau threshold is the super-elastic threshold of the Nitinol driving wire, which only occurs in tension, and prevents additional force from being induced into the medical device tip. The medical device breaks threshold occurs in the proximal section of the medical device. Due to the multiplicity of safety thresholds, the chances of harmful forces being induced in the medical device tip are highly unlikely.

According to some embodiments, breakaway configurations can include a linear motion device with a plurality of guide rails laid in parallel and straight at a predetermined distance. The linear motion device can include a plurality of sliders arranged on each side of the plurality of guide rails and movable on the guide rails. A movable part can be bridged over all of the plurality of guide rails and can be supported by the plurality of sliders and be movable along the plurality of guide rails.

FIG. 19 shows an exemplary linear motion device configured as a slide mechanism 1200 for implementing a breakaway connection according to an exemplary embodiment. Exemplary sliders 126, 226, and 326 were previously described and further features are disclosed below. The slide mechanism 1200 is a linear guide rail with an aluminum pusher 1210 on the treatment side and an aluminum pusher 1211 on the actuator side, two magnets 1212 contained within two steel containers 1213, two plastic or non-ferrous spacers or shims 1214, and a linear bearing 1215. The slide mechanism 1200 is a linear guide rail with an aluminum pusher 1210 on the treatment side and an aluminum pusher 1211 on the actuator side, two magnets 1212 contained within two steel containers 1213, two plastic or non-ferrous spacers or shims 1214, and a linear bearing 1215. The magnets 1212 can be, for example, N52 NdFeB magnets or other types of magnets. Linear guide rails, guideways, and slides are mechanical systems composed of rails and bearings that support and move physical loads along a linear path with a low coefficient of friction. These are generally considered as rolling element or plane bushing types. Common types of guideways and bearings include profiled (square) rails with recirculating ball bearing blocks, guideways for roller bearings, and round rails with recirculating ball bushings or plane bushings. The slides can be saddle, cantilever, or other types of slides. The slide mechanism 1200 can have a plurality of guide rails laid in parallel and straight at a predetermined distance. A plurality of sliders can be arranged on each of the plurality of guide rails and movable on the guide rails, and a movable part that is bridged over all of the guide rails and is supported by the plurality of sliders and movable along the plurality of guide rails.

According to some embodiments, both the actuator side and the treatment side can include a slider mechanism. The actuator side slider mechanism and the treatment side slider mechanism can together constrain the at least one drive wire in at least one or more dimensions, including five or six dimensions. Each bendable section preferably includes at least three driving wires.

With the integration of the magnetic breakaway, the magnetic breakaway configurations described above can be integrated in the slide mechanism 1200 with a single linear rail style bearing and two carriages attached to the same rail. This allows for the two pushers of the actuator and medical device to move freely when decoupled during breakaway, or move together when coupled during normal operation. When decoupling occurs, the linear rail keeps the two pushers aligned, allowing for simple re-attachment, thus normal operation can be resumed.

The medical device can be coupled to the linear guide rail of the slide mechanism 1200 to allow components of the breakaway connection to slide along the guide rail. The linear guide rail can include a guide rail with two or more shafts mounted to the bottom of the breakaway connection.

Magnetic breakaway configurations according to some embodiments can functionally interact with a medical device or continuum robot, robotic or snake catheter assembly with a rotational drive assembly to impart rotational movement to a guide wire of a steerable catheter, endoscope, or other surgical tool.

A steerable catheter apparatus according to some embodiments can include a steerable catheter with distal and proximal ends, a plurality of bending sections, an actuator to push and/or pull push-pull wires, and can include other features.

The push-pull wires can be attached to the distal end of the bending sections on one end and can be attached to actuators on the proximal end of the bending sections on the other end to bend the bending sections.

A tensile force breakaway mechanism can engage at a tensile threshold force equal to or greater than a predetermined value to stop tensile force transmission via the push-pull wires from the actuator to the steerable catheter. A compression force breakaway mechanism can engage at a compression threshold force equal to or greater than a predetermined value to stop compression force transmission via the push-pull wires from the actuator to the steerable catheter.

The tensile force breakaway mechanism can be in the steerable catheter.

The compression force breakaway mechanism can be in the actuator.

The push-pull wires of the tensile force breakaway mechanism can be made of super-elastic material in the form of nickel titanium alloy or Nitinol, wherein the push-pull wires can reach a super-elastic plateau of the super-elastic material in a stress-strain relationship at the tensile threshold force, and the elongation of the push-pull wires in the super-elastic plateau state can stop the tensile force transmission.

The push-pull wires in the super-elastic plateau state can have a maximum elongation equal to or greater than a predetermined operation stroke of the push-pull wires that can correspond to a maximum required operation stroke and the maximum elongation.

The tensile force breakaway mechanism can have irreversible breakpoints, wherein the irreversible breakpoints can physically disengage the push-pull wires from the actuator irreversibly to stop tensile force transmission.

The compression force breakaway mechanism can include permanent magnets.

The actuator can include force sensors to measure push and pull forces on the push-pull wires, motors to push or pull the push-pull wires based on operation commands, a controller to issue the operation command to the motors, and can include other elements or components.

The controller can issue the commands based on metrics computed with force measurements from the force sensors to stop increasing the push or pull force on the push-pull wires.

By separating tensile and compression force breakaway mechanisms for push-pull wires, the magnetic breakaway configurations according to embodiments described herein produce optimized mechanisms for tensile and compression forces with a different principle.

Since failure of the push-pull driving wires with the compression forces basically happen on the force transmission line with the push-pull driving wires, by locating the compression force breakaway mechanism at the actuator, the breakaway mechanism covers all force transmission lines with the push-pull wires between the push-pull wires and the actuator. On the other hand, since the failure of the push-pull driving wires with the tensile forces basically happen either at around the anchoring points or the thin diameter part of the push-pull driving wires in the steerable catheter body, the tensile compression force breakaway mechanisms can be located at the steerable catheter.

By making the push-pull driving wires of super-elastic Nitinol, the tensile breakaway mechanisms can be formed without additional structure from the push-pull driving wires themselves. Therefore, the catheter can be miniaturized with the breakaway mechanism.

By making the elongation of push-pull driving wires of larger than the maximum required operation stroke, the system guarantee to protect the push-pull driving wires from failures. Specifically, the actuator can equip the stroke limiter for maximum operation stroke distance and can prevent out of range for the tensile breakaway mechanism with simple distance limiter.

The irreversible breakaway can achieve the breakaway action with simple mechanical structure without electricity. This can also miniaturize the catheter even with the tensile breakaway mechanism.

By combining the force management with force sensors and motor control with the tensile and compression breakaway mechanisms, the system can reduce situations when the breakaway mechanisms need to operate.

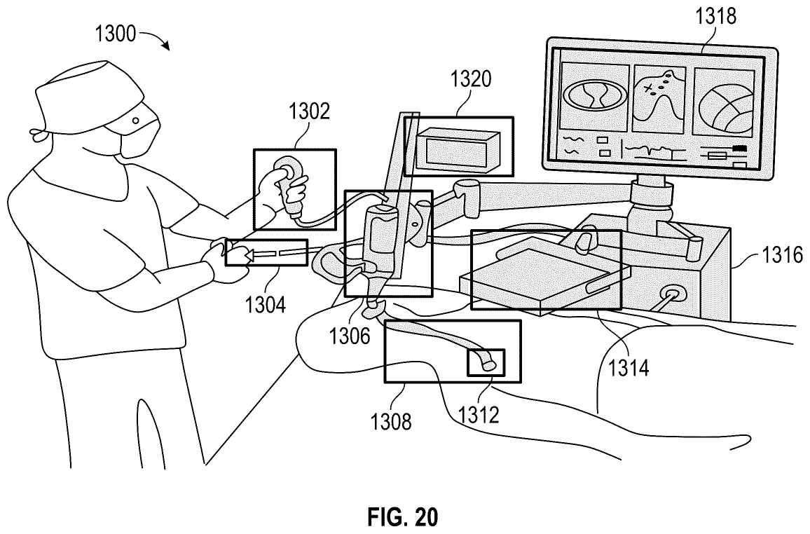

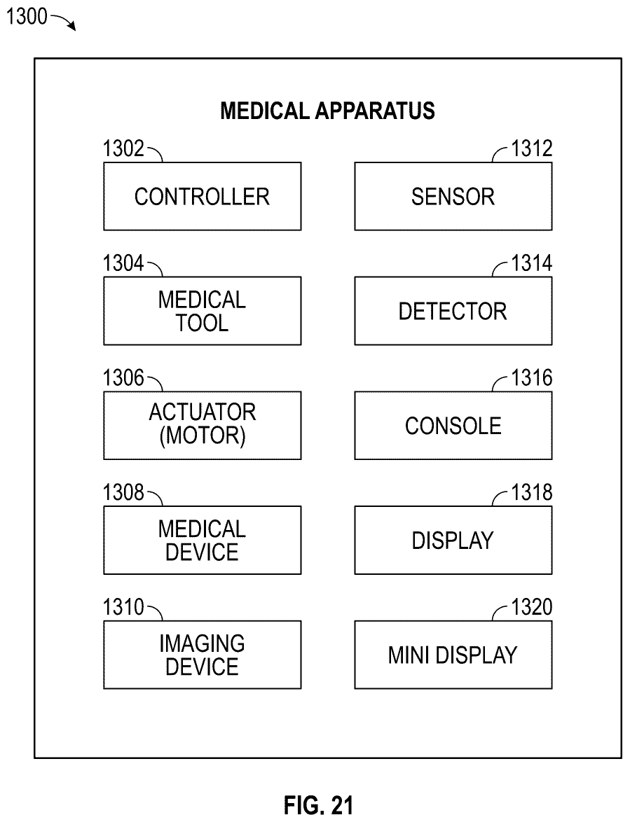

FIG. 20 shows an exemplary medical apparatus 1300 in the form of a robotic catheter assembly or snake configuration that can functionally interact with magnetic breakaway configurations according to some embodiments. FIG. 21 shows a hardware configuration of the medical apparatus 1300.

The medical apparatus 1300 includes one or more of a hand-held controller 1302, a medical tool 1304, an actuator (motor) 1306, a medical device 1308, an imaging device 1310, a sensor 1312, a detector 1314, a console 1316, a display 1318, and a mini display 1320, and can include other elements or components. The medical tool 1304 is referred to as a “biopsy tool” and the medical device 1308 is referred to as a “catheter”, but these are exemplary and one or more of a variety of other types of tools, devices, configurations, or arrangements also falls within the scope of the present disclosure including, for example, a catheter, snake robotic catheter, endoscope, colonoscope, bronchoscope, ablation device, sheath, guidewire, needle, probe, forceps, or the like.

The medical apparatus 1300 according to some embodiments can implement functioning through use of one or more processes, techniques, algorithms, or the like, that can prevent unintended motion while providing better work efficiency to physicians during a medical procedure.

The controller 1302 has a housing with an elongated handle or handle section which can be manually grasped, and one or more input devices including, for example, a lever or a button or another input device that allows a user, such as a physician, nurse, technician, or the like, to send a command to the medical apparatus 1300 to move the catheter 1308. The controller 1302 executes software, computer instructions, algorithms, or the like, so the user can complete all operations with the hand-held controller 1302 by holding it with one hand.

The medical tool 1304 can be a biopsy tool or other type of tool. The actuator 1306 can include one or more motors and drives each section of the catheter 1308. The controller 1302, medical device 1308, console 1314, and other elements are interconnected to the actuator 1306. The controller 1302 includes at least one processor and is configured to control the medical device 1308 through the actuator 1306, and to control the actuator 1306 in accordance with the manipulation by the user.

The medical device 1308 can be configured as a catheter, endoscope or another type of medical device. The imaging device 1310 is a mechanical, digital, or electronic device configured to record, store, or transmit visual images, e.g. a camera, camcorder, motion picture camera, or the like. The sensor 1312 can be an electromagnetic tracking sensor (EM tracking sensor) and is attached to the tip of the catheter 1308. The detector 1314 detects a position of the EM tracking sensor 1312 and outputs the detected positional information to the controller 1302 and/or the console 1318. The controller 1302 receives the positional information of the catheter tip directly from the tracking sensor 1310 or from the detector 1314.

The console 1316 executes software, computer instructions, algorithms, or the like, and controls to display a navigation screen on the display 1318 and other types of imagery or information on the mini-display 1320. The console 1316 can generate a three-dimensional (3D) model of an internal branching structure, for example, lungs or other internal structures, of a patient based on medical images such as CT, MRI, or the like. Alternatively, the 3D model may be received by the console 1316 from another device.

The console 1316 acquires catheter position information from the detector 1314. Alternatively, the console 1316 can acquire the catheter position information directly from the tracking sensor 1312.

The console 1316 generates and outputs the navigation screen to the display 1318 based on the 3D model and the catheter positional information by executing the software. The navigation screen can indicate a current position of the catheter 1308 on the 3D model. By the navigation screen, a user can recognize the current position of the catheter 1308 in the branching structure.

The console 1316 can execute a correction of the acquired 3D model based on the catheter positional information so as to minimize a divergence between the catheter position and a path mapped out on the 3D model.

The display 1318 and/or the mini display 1320 can be a display device configured, for example, as a monitor, an LCD (liquid-crystal display), an LED (light-emitting diode) display, an OLED (organic LED) display, a plasma display, an organic electro luminescence panel, or the like. Based on the control of the apparatus, the navigation screen may be displayed on the display 1316 showing one or more images being captured, captured images, captured moving images recorded on the storage unit, or the like. The mini display 1318 is smaller than the display 1216 and can they can each display similar or other types of imagery and/or information.

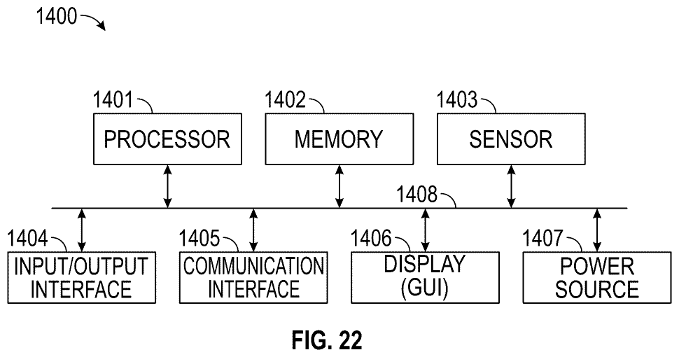

The controller 1302 and/or the console 1316 can include one or more or a combination of levers, keys, buttons, switches, a mouse, a keyboard, or the like, to control the elements of the apparatus 1300 and each has configurational components 1400, as shown in FIG. 22, that include one or more or a combination of a processor 1401, a memory 1402, a sensor 1403, an input and output (I/O) interface 1404, a communication interface 1405, a display 1406, a power source 1407, and can include other elements or components. The apparatus 1400 can be interconnected with medical instruments or a variety of other devices, and can be controlled independently, externally, or remotely by the controller 1402 and/or the console 1414.

The processor 1401, which includes one or more processors, circuitry, or combinations thereof, performs overall control of the medical apparatus 1300, and can execute a program, instructions, code or software stored in the memory 1402 to perform various data processing, computation, algorithmic tasks, or other functions of the medical apparatus 1300. The memory 1402 can store the program, software, computer instructions, information, other data, or combinations thereof. The memory 1402 is used as a work memory. The processor 1401 executes the software developed in the memory 1402.

The sensor 1403 can monitor, measure or detect various types of data of the medical apparatus 1300, and can transmit or send the sensor readings or data to a host through a network. The I/O interface 1404 can interconnect various components with the medical apparatus 1300 to transfer data or information to or from the medical apparatus 1300. The I/O interface 1404 can input the catheter positional information to the console 1316 and can output information for displaying a navigation screen to the display 1316. The communication interface 1405 can interconnect various components with the medical apparatus 1300 to facilitate communication to or from or the medical apparatus 1300.

The display 1406 corresponds to the display 1320 and/or the display 1322 and can present a display to a user to view images, data or other information, and can be configured as an LCD or other type of display. The controller 1302 and/or the console 1314 can perform display control of the display 1406 and control of input of various kinds of setting or default information set by the input/output interface 1404 and the communication interface 1405, and to provide inputs to the medical apparatus 1300.

The power source 1407 provides power to the medical apparatus 1300 to maintain a regulated power supply to the medical apparatus 1300, and can operate in a power-on mode, a power-off mode, and can operate in other modes. The power source 1407 can include a battery contained in the medical apparatus 1300 and can include an external power source such as line power or AC power from a power outlet that can interconnect with the medical apparatus 1300 through an AC/DC adapter and a DC/DC converter, or an AC/DC converter in order to adapt the power voltage from a source into one or more voltages used by components in the medical apparatus 1300.

The components are connected together by a bus 1408 so that the components can communicate with each other. The bus 1408 connects the medical apparatus 1300 to input devices, output devices, communication devices, or other devices. The input devices are configured to enable the user to communicate information and select commands to the medical apparatus 1300, and can include one or more or a combination of a mouse, keyboard, touchscreen, or the like, with keys or buttons with alphanumeric, icon, emoji, or other types of symbols. The output devices are configured to display data or images generated by the medical apparatus 1300, and can include printers, display devices, or other output configurations.

The bus 1408 transmits and receives data between these pieces of hardware connected together, or transmits a command from the processor 1401 to the other pieces of hardware. The components can be implemented by one or more physical devices that may be coupled to the processor 1401 through a communication channel. For example, the controller 1302 and/or the console 1314 can be implemented using circuitry in the form of ASIC (application specific integrated circuits) or the like. Alternatively, the controller 1302 and/or the console 1314 can be implemented as a combination of hardware and software, where the software is loaded into a processor from a memory or over a network connection. Functionality of the controller 1302 and/or the console 1314 can be stored on a storage medium, which may include RAM (random-access memory), ROM (read only memory), magnetic or optical drive, diskette, cloud storage, or the like.

The sensor 1403 can include one or more or a combination of a processor, detection circuitry, memory, hardware, software, firmware, and can include other circuitry, elements, or components. The sensor 1403 can be a plurality of sensors and acquires sensor information output from one or more sensors that detect force, motion, current position and movement of components interconnected with the medical apparatus 1300. The sensor 1403 can include a multi-axis acceleration or accelerometer sensor and a multi-axis gyroscope sensor, can be a combination of an acceleration and gyroscope sensors, can include other sensors, and can be configured through the use of a piezoelectric transducer, a mechanical switch, a single axis accelerometer, a multi-axis accelerometer, or other types of configurations. The sensor 1403 can monitor, detect, measure, record, or store physical, operational, quantifiable data or other characteristic parameters of the medical apparatus 1300 including one or more or a combination of a force, impact, shock, drop, fall, movement, acceleration, deceleration, velocity, rotation, temperature, pressure position, orientation, motion, or other types of data of the medical apparatus 100 in multiple axes, in a multi-dimensional manner, along an x axis, y axis, z axis, or any combination thereof, and can generate sensor readings, information, data, a digital signal, an electronic signal, or other types of information corresponding to the detected state. The medical apparatus 1300 can transmit or send the sensor reading data wirelessly or in a wired manner to a remote host or server. The sensor 1403 can be interrogated and can generate a sensor reading signal or information that can be processed in real time, stored, post processed at a later time, or combinations thereof. The information or data that is generated by the sensor 803 can be processed, demodulated, filtered, or conditioned to remove noise or other types of signals. The sensor 1403 can include one or more or a combination of a force sensor, an acceleration, deceleration, or accelerometer sensor, a gyroscope sensor, a power sensor, a battery sensor, a proximity sensor, a motion sensor, a position sensor, a rotation sensor, a magnetic sensor, a barometric sensor, an illumination sensor, a pressure sensor, an angular position sensor, a temperature sensor, an altimeter sensor, an infrared sensor, a sound sensor, an air monitoring sensor, a piezoelectric sensor, a strain gauge sensor, a sound sensor, a vibration sensor, a depth sensor, and can include other types of sensors.

The force sensor can be located between the actuator and the drive wire according to some embodiments. When a force is induced between the actuator and the drive wire, the sensor can output a voltage to a controller. With programmed software or firmware limits, when a threshold voltage is passed, the controller can stop motion and send a warning signal to the user. This can be referred to as a “motor stop circuit”.

This type of threshold voltage can occur at a lower force than the mechanical breakaway mechanism as it is easier to recover to a normal operational state since no damage or mechanism engagement has occurred. While this electrical sensing can prevent the actuator from inducing more force into the breakaway mechanism, outside factors can still cause additional force to be induced into the catheter. This is when the mechanical threshold takes effect.

The force sensor can also trigger an “auto-relax mode” which can occur before the “motor stop circuit”, but at an upper limit of acceptable operating wire forces. This can be a software algorithm that pauses operation and relieves tension/compression in the wire for better performance and to help prevent higher wire forces from being induced.

Force management for different force thresholds can be implemented according to some embodiments. As illustrated in the force management diagram 600 of FIG. 12, force thresholds for force management purposes can include an auto-relax threshold, a motor stop threshold, a mechanical breakaway threshold, a super-elastic plateau threshold, a catheter breaks threshold, a harmful force threshold, and can include other type of force thresholds.

The auto-relax mode or threshold can occur at an upper limit of wire forces during normal use. The motor stop threshold can stop all actuator motion and can prevent any additional forces from being induced by the actuator to the catheter. The mechanical breakaway threshold is a passive mechanical breakaway threshold, i.e., magnetic breakaway, which engages regardless of electrical signals. The super-elastic plateau threshold is the super-elastic threshold of the Nitinol driving wire, which only occurs in tension, and prevents additional force from being induced into the catheter tip. The catheter breaks threshold occurs in the proximal section of the catheter. Due to the multiplicity of safety thresholds, the chances of harmful forces being induced in the catheter tip are highly unlikely.

The acceleration sensor, for example, can sense or measure the displacement of mass of a component of the medical apparatus 1300 with a position or sense the speed of a motion of the component of the medical apparatus 1300. The gyroscope sensor can sense or measure angular velocity or an angle of motion and can measure movement of the medical apparatus 1300 in up to six total degrees of freedom in three-dimensional space including three degrees of translation freedom along cartesian x, y, and z coordinates and orientation changes between those axes through rotation along one or more or of a yaw axis, a pitch axis, a roll axis, and a horizontal axis. Yaw is when the component of the medical apparatus 1300 twists left or right on a vertical axis. Rotation on the front-to-back axis is called roll. Rotation from side to side is called pitch.

The acceleration sensor can include, for example, a gravity sensor, a drop detection sensor, or the like. The gyroscope sensor can include an angular velocity sensor, a hand-shake correction sensor, a geomagnetism sensor, or the like. The position sensor can be a global positioning system (GPS) sensor that receives data output from a GPS. The longitudinal and latitude of a current position can be obtained from access points of a radio frequency identification device (RFID) and a WiFi device and information output from wireless base stations, for example, so that these detections may be used as position sensors. These sensors can be arranged internally or externally of the medical apparatus 1300.

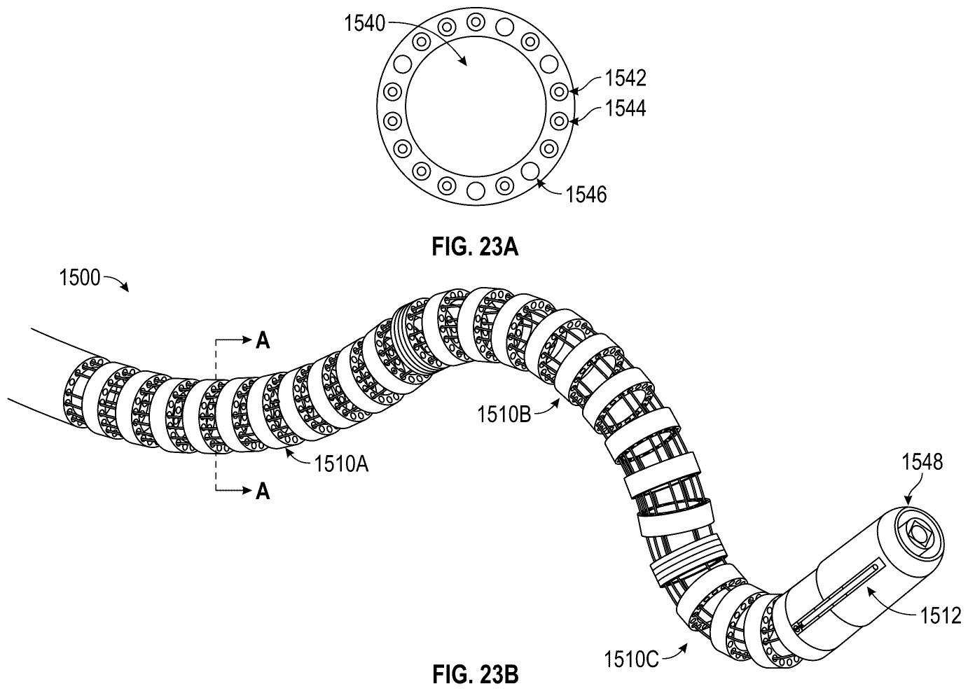

The medical device 1308 according to some embodiments can be configured as a catheter 1500, as shown in FIGS. 23A and 23B.

In FIG. 23A, the catheter 1500 is shown as a flexible medical device that includes a proximal section 910A, a middle section 1510B, and a distal section 1510C. Running proximal to distal through the catheter 1500 is a hollow chamber 1540 that can be used as a working channel for medical procedures. The catheter 1500 includes a plurality of driving wires 1542 and supporting wires 1544 that are each located in lumen 1546 surrounding the central hollow chamber 1540, as shown in the cross-sectional view of FIG. 23B.

Each of the proximal section 1510A, middle section 1510B, and distal section 1510C of the catheter 1500 can be bent by the plurality of driving wires 1542 (driving linear members) as driving backbones. The posture of the catheter 1500 can be maintained by the supporting wires 1544 (supporting linear members) as passive sliding backbones. One or more lumen can be left free to facilitate the use of additional optical fibers or wires to be added to the catheter 1500. The tracking sensor 1310 is attached to the atraumatic tip 1548 of the catheter 1500. At the proximal end of the catheter 1500, the driving wires 1542 are connected to the actuator 1506. The actuator 1306 can include one or more motors and drives each section of the catheter 1500 by pushing and/or pulling the driving wires 1542 in a push/pull or insertion/pulling-out direction.

The controller 1302 can control the catheter 1308 based on an algorithm known as follow-the-leader (FTL) algorithm. By applying the FTL algorithm, the middle section and the proximal section (following sections) of the catheter 708 move at a first position in the same way as the distal section moved at the first position or a second position near the first position.

Breakaway connections according the some embodiments provide compressive breakaway, a rigid support mechanism, reset-ability, precise and repeatable coupling force, high force per volume, as well as numerous advantages over known techniques.

For example, alternative methods rely on slip, which creates wear on the surface in which slipping occurs. Minimal wear occurs with the breakaway configurations described above, where axial magnetic forces may be present but there are no lateral frictional forces.

In instances of repeatable force, the coupling force is dependent on the volume of the magnet and the gap between the magnet and the steel. This makes the force easy to control precisely between parts and create a precise repeatable force between units. This increases the manufacturing cost. Also, with friction-based breakaways, the fore will gradually decrease over time as they wear.

Breakaway configurations according to the embodiments described above account for support after breakaway occurs. In cases where breakaway takes place, the breakaway configurations according to some embodiments remain functional after breakaway occurs because there are sliding members to support the catheter and actuator moving parts separately. This also allows for easy re-engagement of the two after breakaway.

As described above, magnetic breakaway configurations are described that advantageously improve safety and address issues regarding rigidity, force density, and bandwidth.

A breakaway apparatus according to some embodiments includes an actuator, a medical device, and a breakaway connection coupling the actuator and the medical device, the breakaway connection having an actuator portion on the actuator and a medical device portion on the medical device. The actuator portion can include an actuator support, an actuator side ferromagnetic block, and a slider element. The medical device portion can include a medical device support, a medical device side ferromagnetic block, and a slider element. At least one of the actuator side ferromagnetic block and the medical device side ferromagnetic block includes a magnet, and the actuator is coupled to the medical device by a magnetic coupling force. The actuator will decouple from the medical device when a decoupling force is greater than a predetermined amount.

The actuator portion can include a spacer attached to the actuator side ferromagnetic block and facing the medical device ferromagnetic block, the spacer defining a gap between the actuator side ferromagnetic block and the medical device ferromagnetic block. Both the actuator side ferromagnetic block and the medical device side ferromagnetic block can include a magnet.

The slider element in the actuator portion and the slider element in the medical device portion can be a single slider element that is connected to both the actuator support and the medical device support.

The actuator can include a motor, a drive, or a motorized drive assembly. The actuator can include a rotational drive assembly to impart rotational movement to the plurality of drive wires. The plurality of drive wires can include a monolithic material wire configured for both push and pull motions.

The medical device can be a catheter, endoscope, colonoscope, bronchoscope, or ablation device. The medical device can be a steerable catheter with distal and proximal ends and a plurality of driving wires. The steerable catheter can include a plurality of bendable sections located at a distal end of the steerable catheter, one of the plurality of drive wires passing through each bendable section. The driving wires are push-pull wires can have a distal end attached to a distal end of one of the plurality of bendable sections and a proximal end attached to the medical device portion.

A tensile force breakaway mechanism can engage at a tensile threshold force presented on at least one of the plurality of driving wires, which is equal to or greater than a predetermined value to stop tensile force transmission through the actuator portion to the steerable catheter. The tensile force breakaway mechanism can be in the steerable catheter. The plurality of driving wires can include super-elastic material in the form of nickel titanium alloy. The plurality of driving wires reach a super-elastic plateau of the super-elastic material in a stress-strain relationship at the tensile threshold force, and an elongation of the plurality of driving wires in the super-elastic plateau state is configured to stop the tensile force transmission. The plurality of driving wires in the super-elastic plateau state can have a maximum elongation equal to or greater than a predetermined operation stroke distance of the plurality of driving wires that corresponds to a maximum operation stroke distance and the maximum elongation.

A compression force breakaway mechanism can engage at a compression threshold force on at least one of the plurality of driving wires to stop compression force transmission via the at least one driving wire from the actuator to the steerable catheter. The compression force breakaway mechanism can be in the actuator. The compression force breakaway mechanism can include permanent magnets.

The slider element on the actuator portion and the slider element on the medical device portion together constrain the plurality of drive wires in at least one dimension. The slider element on the actuator portion and the slider element on the medical device portion together constrain the plurality of drive wires in five dimensions.

The breakaway apparatus can include at least one force sensor coupled to the steerable catheter or medical device portion to measure push and pull forces on the plurality of driving wires, at least one motor to push or pull the plurality of driving wires based on operation commands, and a controller to issue the operation command to the at least one motor. The controller can issue the commands based on metrics computed with force measurements from the force sensors to stop increasing the push or pull force on the push-pull wires. The actuator can exert linear force provided by a motor. The linear force can act through the breakaway apparatus exerting itself onto the medical device.

The actuator portion and the medical device portion can be offset from each other in a direction of the magnetic coupling force. An actuator pusher can be coupled to the actuator. The medical device can include a medical device wire pusher. The actuator ferromagnetic block and the medical device ferromagnetic block are configured to permeate magnetic flux and create a bond between each other.

A system according to some embodiments can include an actuator, a steerable device having a plurality of driving wires, and a plurality of breakaway connections coupling the actuator and the steerable device, each breakaway connection having an actuator portion on the actuator and a steerable device portion on the medical device.

The actuator portions can each include an actuator support, an actuator side ferromagnetic block, and a slider element. The steerable device portions can each include a device support, a steerable device side ferromagnetic block, and a slider element. The at least one of the actuator side ferromagnetic block and the device side ferromagnetic block can include a magnet. A tensile force breakaway mechanism can engage at a tensile threshold force on at least one of the plurality of driving wires which is equal to or greater than a predetermined value to stop tensile force transmission through the actuator portion to the steerable device. A compression force breakaway mechanism can engage at a compression threshold force on at least one of the plurality of driving wires to stop compression force transmission through the actuator to the steerable device. The system can include at least six breakaway connections and the steerable device can include at least three driving wires. The actuator is coupled to the medical device by a magnetic coupling force, and the actuator will decouple from the steerable device when a decoupling force is greater than a predetermined amount.

Both the actuator side ferromagnetic block and the steerable device side ferromagnetic block can include a magnet. The slider element in the actuator portion and the slider element in the steerable device portion can be a single slider element that is connected to both the actuator support and the steerable device support. The actuator can include a motor, a drive, or a motorized drive assembly.

The system can also include any combination of additional features as described above for the breakaway apparatus.

Breakaway mechanisms according to the present disclosure provide various advantages over related art with respect to minimal wear, repeatable force, accounts for support after breakaway occurs, as well as other advantages.

With respect to minimal wear, related art methods rely on slip, which creates wear on the surfaces in which slipping occurs. With breakaway mechanisms according to the present disclosure, it is possible for there to be no lateral frictional force, however solely axial magnetic force.

With respect to repeatable force, the coupling force is dependent on the volume of the magnetic and the gap between the magnet and the steel. This makes the force easy to control precisely between parts and between breakaways of the same part. When dealing with slip designs, tight tolerances should be made to create a precise repeatable force between units. This increases the manufacturing cost. Also, with friction caused breakaways, the force will gradually decrease over time as they wear.

To account for support after breakaway occurs, breakaway mechanisms according to the present disclosure can be functional after breakaway occurs in which there are sliding members to support the catheter and actuator moving parts separately. This allows for easy re-engagement of the two after breakaway.

Features of breakaway mechanisms according some embodiments include compressive breakaway, rigid support mechanism, reset-ability, precise and repeatable coupling force, high force per volume, and there can be other features.

A steerable catheter apparatus according to some embodiments can include a steerable catheter with distal and proximal ends, a plurality of bending sections, an actuator to push and/or pull push-pull wires, and can include other features.

The push-pull wires can be attached to the distal end of the bending sections on one end and can be attached to actuators on the proximal end of the bending sections on the other end to bend the bending sections.

A tensile force breakaway mechanism can engage at a tensile threshold force equal to or greater than a predetermined value to stop tensile force transmission via the push-pull wires from the actuator to the steerable catheter. A compression force breakaway mechanism can engage at a compression threshold force equal to or greater than a predetermined value to stop compression force transmission via the push-pull wires from the actuator to the steerable catheter.

The tensile force breakaway mechanism can be in the steerable catheter.

The compression force breakaway mechanism can be in the actuator.

By separating tensile and compression force breakaway mechanisms for push-pull wires, advantages of these features can be applied to an optimized mechanism for tensile and compression forces with the different principle.

Also, since the failure of the push-pull driving wires with the compression forces basically happens all force transmission lines with the push-pull driving wires, by locating the compression force breakaway mechanism at the actuator, the breakaway mechanism covers all force transmission line with the push-pull wires to the connection part between the push-pull wires and the actuator. On the other hand, since the failure of the push-pull driving wires with the tensile forces basically happens either at around the anchoring points or the thin diameter part of the push-pull driving wires in the steerable catheter body, the tensile compression force breakaway mechanisms can be located at the steerable catheter.

The push-pull wires of the tensile force breakaway mechanism can be made of super-elastic material in the form of nickel titanium alloy or Nitinol, wherein the push-pull wires can reach a super-elastic plateau of the super-elastic material in a stress-strain relationship at the tensile threshold force, and the elongation of the push-pull wires in the super-elastic plateau state can stop the tensile force transmission.

By making the push-pull drive wires of super-elastic Nitinol, the tensile breakaway mechanism can be formed without additional structure from the push-pull drive wires themselves. Therefore, the catheter can be miniaturized with the breakaway mechanism.

The push-pull wires in the super-elastic plateau state can have a maximum elongation equal to or greater than a predetermined operation stroke of the push-pull wires that can correspond to a maximum required operation stroke and the maximum elongation.

By making the elongation of the push-pull drive wires larger than the maximum operation stroke distance, the apparatus can guarantee to protect the push-pull drive wires from failure. Specifically, the actuator can equip the stroke limiter for maximum required operation stroke and can prevent out of range for the tensile breakaway mechanism with a simple distance limiter.

The tensile force breakaway mechanism can have irreversible breakpoints, wherein the irreversible breakpoints can physically disengage the push-pull wires from the actuator irreversibly to stop tensile force transmission.

The irreversible breakaway can achieve the breakaway action with simple mechanical structure without electricity. This can also miniaturize the catheter even with the tensile breakaway mechanism.

The compression force breakaway mechanism can include permanent magnets.

The compression breakaway mechanism with permanent magnets can achieve a reliable breakaway with less wear while having enough breakaway threshold force.

Moreover, since the compression breakaway mechanism is in the actuator, the apparatus can hide the permanent magnet from the outside and increase compatibility to the equipment sensitive to the magnet and safety.

The actuator can include force sensors to measure push and pull forces on the push-pull wires, motors to push or pull the push-pull wires based on operation commands, a controller to issue the operation command to the motors, and can include other elements or components.

The controller can issue the commands based on metrics computed with force measurements from the force sensors to stop increasing the push or pull force on the push-pull wires.

By combining the force management with force sensors and motor control with the tensile and compression breakaway mechanisms, the apparatus can reduce situations when the breakaway mechanisms need to operate.

Breakaway mechanisms according to some embodiments are incorporated into a medical device in the form of a robotic or snake catheter assembly or other type of medical assembly that has a rotational drive assembly to impart rotational movement to a guide wire of a medical instrument such as a catheter, an optical catheter, or other flexible and thin tubular instrument made of medical grade material that can be inserted through an opening in a bodily lumen (e.g. a vessel) to perform a broad range of medical functions. A fiber optic catheter is an example of an optical catheter that includes a flexible sheath, a coil, and an optical probe or imaging core contained within the coil. The catheter can include a ‘guide catheter’ which functions similarly to a sheath. The drive assembly is releasably connected to the catheter and a breakaway mechanism can be used so the drive assembly disconnects from the catheter in response to a coupling force.

While the present disclosure has been described with reference to exemplary embodiments, it is to be understood that the disclosure is not limited to the disclosed exemplary embodiments. The scope of the following claims is to be accorded the broadest interpretation so as to encompass all such modifications and equivalent structures and functions.

1. A breakaway apparatus comprising:
an actuator;
a medical device; and
a breakaway connection coupling the actuator and the medical device, the breakaway connection having an actuator portion on the actuator and a medical device portion on the medical device,
the actuator portion comprising:
an actuator support;
an actuator side ferromagnetic block; and
a slider element,
the medical device portion comprising:
a medical device support;
a medical device side ferromagnetic block; and
a slider element,
wherein at least one of the actuator side ferromagnetic block and the medical device side ferromagnetic block comprises a magnet, and
wherein the actuator is coupled to the medical device by a magnetic coupling force, and the actuator will decouple from the medical device when a decoupling force is greater than a predetermined amount.

2. The breakaway apparatus according to claim 1, wherein the actuator portion further comprises:
a spacer attached to the actuator side ferromagnetic block and facing the medical device ferromagnetic block, the spacer defining a gap between the actuator side ferromagnetic block and the medical device ferromagnetic block.

3. The breakaway apparatus according to claim 1, wherein both the actuator side ferromagnetic block and the medical device side ferromagnetic block comprise a magnet.

4. The breakaway apparatus according to claim 1, wherein the slider element in the actuator portion and the slider element in the medical device portion is a single slider element that is connected to both the actuator support and the medical device support.

5. The breakaway apparatus according to claim 1, wherein the actuator comprises a motor, a drive, or a motorized drive assembly.

6. (canceled)

7. The breakaway apparatus according to claim 1, wherein the medical device comprises a catheter, endoscope, colonoscope, bronchoscope, or ablation device.

8. The breakaway apparatus according to claim 7, wherein the medical device is a steerable catheter with distal and proximal ends and a plurality of driving wires.

9. The breakaway apparatus according to claim 8, wherein the plurality of driving wires comprise a monolithic material wire configured for both push and pull motions.

10. The breakaway apparatus according to claim 8, wherein the steerable catheter further comprises a plurality of bendable sections located at a distal end of the steerable catheter, at least one of the plurality of driving wires passing through each bendable section.

11. The breakaway apparatus according to claim 10, wherein the driving wires are push-pull wires having a distal end attached to a distal end of one of the plurality of bendable sections and a proximal end attached to the medical device portion.

12. The breakaway apparatus according to claim 10, wherein a tensile force breakaway mechanism engages at a tensile threshold force presented on at least one of the plurality of driving wires, which is equal to or greater than a predetermined value to stop tensile force transmission through the actuator portion to the steerable catheter.

13. (canceled)

14. The breakaway apparatus according to claim 12, wherein the plurality of driving wires comprise super-elastic material in the form of nickel titanium alloy.

15. The breakaway apparatus according to claim 14, wherein the plurality of driving wires reach a super-elastic plateau of the super-elastic material in a stress-strain relationship at the tensile threshold force, and an elongation of the plurality of driving wires in the super-elastic plateau state is configured to stop the tensile force transmission.

16. The breakaway apparatus according to claim 15, wherein the plurality of driving wires in the super-elastic plateau state have a maximum elongation equal to or greater than a predetermined operation stroke distance of the plurality of driving wires that corresponds to a maximum operation stroke distance and the maximum elongation.

17. The breakaway apparatus according to claim 10, wherein a compression force breakaway mechanism engages at a compression threshold force on at least one of the plurality of driving wires to stop compression force transmission via the at least one driving wire from the actuator to the steerable catheter.

18-19. (canceled)

20. The breakaway apparatus according to claim 10, wherein the slider element on the actuator portion and the slider element on the medical device portion together constrain the plurality of drive wires in at least one dimension.

21. The breakaway apparatus according to claim 10, wherein the slider element on the actuator portion and the slider element on the medical device portion together constrain the plurality of drive wires in five dimensions.

22. The breakaway apparatus according to claim 1, further comprising:
at least one force sensor coupled to the steerable catheter or medical device portion to measure push and pull forces on the plurality of driving wires;
at least one motor to push or pull the plurality of driving wires based on operation commands; and
a controller to issue the operation command to the at least one motor.

23. The breakaway apparatus according to claim 22, wherein the controller is configured to issue the commands based on metrics computed with force measurements from the force sensors to stop increasing the push or pull force on the push-pull wires.

24-25. (canceled)

26. The breakaway apparatus according to claim 1, wherein the actuator portion and the medical device portion are offset from each other in a direction of the magnetic coupling force.

27-29. (canceled)

30. A system comprising:
an actuator;
a steerable device having a plurality of driving wires; and
a plurality of breakaway connections coupling the actuator and the steerable device, each breakaway connection having an actuator portion on the actuator and a steerable device portion on the medical device,
the actuator portions each comprising:
an actuator support;
an actuator side ferromagnetic block; and
a slider element,
the steerable device portions each comprising:
a device support;
a steerable device side ferromagnetic block; and
a slider element,
wherein at least one of the actuator side ferromagnetic block and the device side ferromagnetic block comprises a magnet,
wherein a tensile force breakaway mechanism engages at a tensile threshold force on at least one of the plurality of driving wires which is equal to or greater than a predetermined value to stop tensile force transmission through the actuator portion to the steerable device, and
wherein a compression force breakaway mechanism engages at a compression threshold force on at least one of the plurality of driving wires to stop compression force transmission through the actuator to the steerable device.

31-58. (canceled)
